SEQUENCE LISTING (11 sequences)

SEQ ID NO: 1 - DNA, 7518 bp, Bos taurus
  misc_feature (1)..(7518): “n” can be any nucleotide
cctgaagactctgaaatcaccataacagtatggttttcttttgcaggagaaacaaataaaaagagctgagcatgactgtcttctattgttctaagcatggccctgcaaggaactgagtgttccttccaaagacagagtgccataagtaacaaggaagagaacttgcaatgcttgacttggtgtctaaaaatcgtatttactttgtctctaatgattctatcaaataataaatgactggaatgatgcaagaaagtgggaagtggaattccggtatggaatggctccaaagtcaaagagttacaaaagaatgtgatcactggcccatgatttctgccccgaggaaggtagaagaagaaatgtggacatcacagccccactctgtggagttgcccaagggtatgttcagggctgatgactgtagatggttgacagcatcccctctccccaccatgtctactcctggaaccgagtcagctaagccagctcagggacctagactaccccaacctcagggaaccccaactttcgaaggagtgtaagttctgttgagaacatttaaatactgcttagctttgccttgcataaaaagcaaacacagcaatcctttagacaggtatccaccctacatgttcacctcattacacctacaggaaataaaatatgtcccagacccagaaagaggcatcatcctatttccgagaggttctactctaagacagggtgtctcaggtggctcagtggtaaagaatcccctgcaattcaggagacgtgggttcaatccctggatcaggaagatcccctggagaaggaaatctccacgtactccagtactcttccctggaaaatcccacacacagaggagcctgaggggctacaacccatggcgtcactaaagagtcagacacaaacttggtgactaaacaccaacaacacatctaagaccgtggttcccagaccacagtgtcatcctcagctggtgtgcttccgcctgggccagtgctcttctttctaacgtcctgctcatagttccctgagtcttgtaacaaattggaaatgcttccccctccttcttctcaggattactacttgagcacaagtcccagatccttccagactctaatttaccatcccccctcgtctcagttggcaagtctctgtgatttgcttgcatctgtcttggagatcacttccctgtgtctccaatgcatggctcacattcctgctctcctgtgaacactccaggcaagaatcctgaggtggtgctcctgggctgggccttacttgccctcctgctgtcttcaggcttccagcccagggacccaccgtcaggaagagcagttccatgtcctctgggtccccaccatctctgacatattacacatgcctcacccctcctcctctgggaaaacagtaacctttgaaatttccctttatgatgaagaggcagagatggcacaaggagttccagacacgccatccctgtgagagctcctgcatgaaatttcctcccaagattatcttcctgaactcacagaataaagctcagtcctgcttttctgattgatacagctattctctgacatcatacactccagcattcttgtgataaatcagctttctctgtatccttcctgaaccttctatcctgataatttaacagacatctggacttttcctgttcatttttatggtgtcacttctacacgatatcatagcaaaattcctcactgtagttcaaccttcctgggattgcaatgacctctcaatatgacctactgttataactatcaacagcttctgaccatgaatacacttgaagctggaagactcccttttccagggctgaatctaccctcttgtgtgtactggaggtttgattttcctgagttttatctccttcctttcctgctgctgtaccctgaggtcctagcttcatcctgagctgtctcttataaggtacccgttctgctgtttcatttgccctgttgctcgcattctttactcaactccctttttctattgttagtctttggaattcccaggtggctcaggtggtaaagaatccacttgccctgcaagagacccagattcgatccctgggtcagggagctccgcaggagaagacaatggagtactcttgcctggtattcttgcctgccactccagtattcttgcctggagaattccatggagagaggagcctggtgggttacagtccatggggtcccaaagagtcggacaaaactgagcaactaagcacacacacacttcacagcaaaagtcagtacattacgatggggggtgaaaaacacatttgtctcctggagtgagactagaaacgggcaggaaaagcccaatatgctacattctgaaagttgccagattgcacaatctaggaaaattcgatgtcagtgaagaataacactgctgttttgaatgctgtgttctggtcaggaggatttcccaaactttttttccttctttctttttttttgcttccaccccagttctcatttgctccactagccaagtgagagtatataaagcaccggccccgtctcccaggcaggcagcacaggcagctcagcttcaccaggagcctcagcaggagggcacggccacaggtgaggtgctagaactctccaacacttttcctcttcggagactctctcttcagcagcattcttgcgctgcagcccaactctgcttccttcctgaatctactgttctgaccattagaatccaccagattgagcacttcagggagtagggctcatcttgtctgcatcttctgtgcaggcagcgatggggtgagcacgcaggccacagacacatgtgcctcgttcacctcgtctcgtatcacagagaggcagcatgaacacactcctcttgcctttgggaaacttgcagtgcagctgggtctcagggctgatagaggatgactggactggaaagtggtttatgctaaaagcacgttgcaatccttcacacaggaaatcattgggattccaagatttcatatggaaataagagctggatcctctgtgttacaacctatcgtctgtctactgagataaaattcagaggggtttatgttcggaatgtaagagtgtatccacattacaactcagccccaagacctgtcattcttgattgactccgctcatctctctgttgcaggatgaacctttccacgggcatcattttctgcttcctgatcctgggcgtcagcagccagagatgggggacattcctcaaggaagctggtcaaggtaaggaccaaaggatgggccaggggaggctgtgtctgcttccccaggattgacctgagcagaggacacatccccacagggcaaaggccacaggtgggcagaaaagaagcttagttttcatggtagcacttcccgaagcttttctggccagctttgcactcttttaggggatccccaagcccgaggtcacataaagtttgggccccaactttcagcaggagtgaggaagacatctggggggcaaggtatctgttgccaaaataccagtaaggctctgctaccgcctcgtgggcaactagagatggctcatttccaagtctcctgtagccatgaagtgggtgcaaccgctgaatacttataaataaaatacttgattttttagtagctgcccaggactgtctaagagctttatatgcaggaatcgactcgttttccccctcagggtttaatccttgagtcctgcaatgtagggaccatcaccccttatcagagaacctgctgccccaagagattaagatagggtccaacatcctccagcagagcaggattgaacccagcatcctgagaccttgctgttgacttcggcccttctactgcctcccagacaagagtacacgtggagggtgaggggtctgtgaacacgcatcctggtctttatctgagcagatggcagagagtgggggttgctgcctttggaaggaaacccgatagagctcccctccccacagtaaatggcagcatgagtttccttgatgatggttctgctgaggctgagacctggcgagaatcctatagcaagagatatagacctcactagccagagcaaactggccataatttatttcccaaaactatttggtgttattatttttctgtgataattgctgaataattgttttaagcatttgttcttaattccatctaaattcacacaggcccagataaaagtatcttttcatctcttaggtcagtgttgttcaaggggcactctaggatgacttgcatgagaattaaccgtggtctgggtgctttgtggaatgcaggtgcctggatccacacacagtcccttccctgaaccacagtccctggggctgtctgcaaatctgtccattattgagcaccccacttgattttgtgcacagtaaacactgagaaccactaccttgttttgcacccaagggacaaatatgtcgtgcatttggaagcacttattaaacaactctagactccagggaactatttaaatctgtaactcagggtgcatagctatagtaagaatatcatagccctcaaccaaactatttttctgaacagtggaaatagctaacacctaaaataaagataagttatctcatagagatattacataaactattattataatccatgttatattttcctcttccctaatgagctaatcatttaaacctttgccattttattctatttaggttgggttttctgtccatgcctccctgatctccatccaactttatttatttttttgccctactcttctaaggaccagagaggtgatagtatagtgagcaccgacaatgttccataaactcaacctgtatttcctcagttcttctgcataaccaccctgagggaggcattactcctccattttactggagaggacactgaactttagagctggtgggtcagttgccctttttctgcatctgattaccctgtttcttcaaagccctcttagggagctcacctttatcacctgctgatttaattctgacggttgcccatgtgcaaacatgccctgagtattcagatgtactcaggcccgagttagtccccagggctggatttctccccttgaccagctgggagtatcctatatccacagcctttctcagtatcgtcattctcaagctctgatcagagcctctcctgcgtctttccaggtggaggttcattgtataagcaaacatcccttaaagaaagcattgaccgcttcttcacagacatcacacacctccagaaacaaagttctaacagacttagaatgaaatcaaacagaataaaccttgcatcaagtgtgatactcacaacttcagatcagggaaggaagtgagaagtaaagaagtattcatttcaagccaataaaataatctccaagggcttggtcgaaggctgaaacctaaaatcagtgggaggaaatgatttatttctctttcaccaaaacatgatcacattcatatcatcattttcttttcttcccaggggctaaagacatgtggagagcttaccaagacatgaaagaagccaactacaggggtgcagacaaatacttccacgcccgtggaaactatgacgctgcccgaaggggacctgggggtgcctgggctgctaaagtgatcaggtaccagggtccctggggatgcagggatgggtgagcagagcttggctgcctaggacaacctggaagggcnaagccttggagaactttcctgtaggctgtgngccctcntcctcttacccaccttcctgctctgtgcccactgtgaagtctgaggggctgaagagcagagcaacttggtgggacaggcgactctccacccttnctctatgggtgctgttcacccagcacagggctgaggtgggctgagcctgaggagcctcagggttgtagcccctctttcnttggctcctctcagagtcattgatcccttggaaagaggagagatggggagggtggggctgtggctcatagtcctggattaatcccctccgtgccctcttcctttccagtaacgccagagagactattcagggaatcacagaccctctgtttaagggtatgaccagggaccaggtacgggaggattcgaaggccgaccagtttgccaacgaatggggccggagtggcaaagaccccaaccacttcagacctgctggcctgcctgacaagtactgagctgcctctctctctgctcaggagatggggctgtgagtccccaaggacagggacactgacctagagagttctctgtcctcagaaggaagcagatctaataaatgcttaagagatggaatactgagactgtgtgtcattcttggtataaggacagcctgttagttccaggactgatggccggacaccgacgtgaaggctgagcctgtgcctgtgtgtttggttctggcacacaatctcagcatcattcaggacagacgccctctgcagccttccctaatcagacccgccccctccccagacccctctggtgacacgggggccatttccaggcccttcactgtcaggccttctcactccctgccgttgtgtcctgtccccttctctgtccccaggtctagtcccctagcctgtcctctgtgctctctgtgtggggcatggacacaggaggactggatggtggaatcctgctccagaaactgccacctggatctcctgttcatttctcagcagcacctacaagtacaactatgagccagtttctgtctgtgcatccggaactgcctccagtgctgttcccttccctctcttttccttgccttatacaagttcccaggaacaaacatgtcaaggagtggaggaataatggcaacatgaaaattcagagccaggcgcctttgtttgccttggatatgattcatgtcctcgagaggaagtcgttttcccctcctggtcctttctcaacccagggaagccagcagcagttactttttattgaggaaaacagtgtctcttatggaagggagttgggtctgttagagcacaggaattatgagtgactctgtgagtcataacaatgctgaatatgtaaacgcatacatacacataaataatgcacatgaattatagagattatgataaaataaaaattgataaatgtatcagaaccacaagcagaaattcataatggaaaataaaagggtgtatcatgaataaagtcataatggattcagtaatcttcatgttccatattccatctgttgttgctgttgttcagtcactcagtcatgttgactcttagggaccccatggactgcagcatgccaagtttccctgtccttcactatcacttggagtttgctcaaactcatgtccattgagtctgtgatgccattcaaccacctcatcctctgtggcctccttgtcctcctgccgtcagtctttcccagcgtaagggtcttttccagtgagtcagctgtttgcagcagttggctaaagaatggagcttcagcatcagtctttccaatcaat

SEQ ID NO: 2 - protein, 10 aa, Bos taurus
MWGLTKFEAG

SEQ ID NO: 3 - protein, 10 aa, Bos taurus
MWGTFLKEAG

SEQ ID NO: 4 - protein, 10 aa, Homo sapiens
GWLTFLKAAG

SEQ ID NO: 5 - DNA, 27 bp, Bos Taurus
gacatgtggmgagcctactcygacatg

SEQ ID NO: 6 - protein, 9 aa, Bos Taurus
DMWRAYSDM

SEQ ID NO: 7 - protein, 8 aa, Bos Taurus
SGKDPNHF

SEQ ID NO: 8 - DNA, 27 bp, Bos Taurus
taagggtacgaccagtggccagggtca

SEQ ID NO: 9 - protein, 10 aa, Bos Taurus
FKGTTSGQGQ

SEQ ID NO: 10 - protein, 10 aa, Bos Taurus
GKFAWEGMTL

SEQ ID NO: 11 - protein, 10 aa, Bos Taurus
DAAQRGPQQA

(19) United States
(12) Patent Application Publication
(10) Pub. No.: US 2005/0107315 A1
(43) Pub. Date: May 19, 2005

(54) Mammary-associated serum amuloid a3 promoter sequences and used for same

(75) Inventors: Thomas L. McDonald, Omaha, NE (US); Marilynn A. Larson, Lincoln, NE (US); Annika Weber, Omaha, NE (US)

Correspondence Address: MCKEE, VOORHEES & SEASE, P.L.C.;ATTN: UNIVERSITY OF NEBRASKA MEDICAL CENTER, 801 GRAND AVENUE, SUITE 3200, DES MOINES, IA, 50309-2721, US

(73) Assignee: Board of Regents of the University of Nebraska, Lincoln, NE (US)

(21) Appl. No.: 10/494,484

(22) PCT Filed: Nov. 20, 2002
(86) PCT No.: PCT/US02/37066

Related U.S. Application Data
(60) Provisional application No. 60/333,261, filed on Nov. 21, 2001.

(51) Int. Cl. A61K 48/00; A61K 38/17; C07H 21/04; C12N 9/64

(52) U.S. Cl. 514/44; 435/69.1; 435/226; 435/320.1; 435/325; 514/12; 536/23.2

(57) ABSTRACT

Novel colostrum associated mammalian Serum Amyloid A (SAA) promoter sequences are disclosed. These promoters can be used in transgenic protocols for tissue specific expression and expression constructs, vectors and host cells are disclosed. Also the regulatory features of these promoters in colostrum associated SAA productions are exploited to treat disease states associated with or influenced by colostrum associated SAA production.

### FIELD OF THE INVENTION

The present invention relates to newly identified polynucleotides and their variants, their production and uses, as well as agonists and antagonists, of these polynucleotides and their uses. In particular, the invention relates to promoter polynucleotides from mammary associated serum amyloid 3 (hereinafter referred to as “MAA” ) as well as their variants and uses of the same in treatment of disease and recombinant nucleotide techniques.

### BACKGROUND OF THE INVENTION

Several scientific or patent publications are referenced in this patent application to describe the state of the art to which the invention pertains. Each of these publications is incorporated by reference herein, in its entirety.

Mammals respond to tissue injury, trauma or infection by executing a complex series of biological reactions in an effort to prevent further tissue damage, to initiate repair of damaged tissue, and to isolate and destroy infective organisms. This process is referred to as the inflammatory response, the early and intermediate stages of which are referred to as the acute phase response.

The acute phase response involves a wide variety of mediators, including cytokines, interleukins and tumor necrosis factor. It also involves a radical alteration in the biosynthetic profile of the liver. Under normal circumstances, the liver synthesizes a range of plasma proteins at steady state concentrations. Some of these proteins, the “acute phase” proteins are induced in the inflammatory response to a level many times greater than levels found under normal conditions. Acute phase proteins are reviewed by Steel & Whitehead (Immunology Today 15: 81-87, 1994).

One of the massively induced acute phase proteins is Serum Amyloid A (SAA). SAA actually comprises a family of polymorphic proteins encoded by many genes in a number of mammalian species. SAAs are small apolipoproteins that accumulate and associate rapidly with high-density lipoprotein 3 (HDL3) during the acute phase of the inflammatory response. Most SAA isoforms are induced in response to inflammation; however, certain SAAs (e.g., human SAA4) appear to be constitutively expressed or minimally induced in the inflammatory response.

The liver has been considered the primary site of SAA production. However, SAA production outside the liver has been found, on a limited basis. For instance, expression of SAA mRNA has been reported in human atherosclerotic lesions and in human cultured smooth muscle cells and monocyte/macrophage cell lines (Meek et al., 1994; Urieli-Shoval et al., 1994; Yamada et al., 1996), and a unique isoform of SAA (SAA3) is produced by rabbit synovial fibroblasts (Mitchell et al., J. Clin. Invest. 87: 1177-1185, 1991). More recently, it was discovered that SAA mRNA is widely expressed in many histologically normal epithelial tissues, including tissues of stomach, intestine, tonsil, breast, prostate, thyroid, lung, pancreas, kidney, skin and brain neurons (Urieli-Shoval et al., J. Histochem. Cytochem. 46: 1377-1384, 1998). The role of SAA in such tissues has not been elucidated, nor has it been determined if the SAAs present in those tissues are the same isoforms as those found in serum, or if they represent additional isoforms of SAA.

A mammary Serum Amyloid A (SAA) protein has been identified which was isolated and purified from mammalian colostrum and is produced by ductal epithelial cells of the mammary gland. This protein and nucleic acids encoding the same are disclosed in WO 01/31006 the disclosure of which is hereby incorporated by reference.

### SUMMARY OF THE INVENTION

The present invention relates to mammary associated amyloid A 3 (MAA) and in particular MAA promoter polynucleotides, recombinant materials and methods for their production. In another aspect, the invention relates to methods for using such polynucleotides, including transgenic protocols to provide for temporal and spatial expression of recombinant polynucleotides operably linked thereto. In yet another aspect the invention relates to methods for identifying agonists and antagonists using the materials provided by the invention, and for treating microbial infections and conditions associated with such infections, such as assays for detecting MAA driven promoter expression or activity.

According to the invention, an isolated nucleic acid molecule that encodes a mammalian colostrum SAA promoter is provided. In a preferred embodiment, the nucleic acid molecule comprises a promoter from bovine colostrum SAA sequence. In an even more preferred embodiment the invention comprises a sequence from SEQ ID NO: 1 or its conservatively modified variants.

The invention also comprises nucleic acid constructs wherein said promoter is operably linked to a gene of interest, replication and expression vectors incorporating these constructs, and host cells transformed with these vectors.

In yet another embodiment, agonists or antagonists of the MAA promoter can be identified by assays herein and used as pharmaceutical compositions to stimulate or inhibit MAA production to aid in treatment of diseases associated with the teats or other mammary tissue of animals such as mastitis or to model disease states. For example, as disclosed herein, the colostrum associated SAA promoter is induced by prolactin as well as LPS. Thus one could administer prolactin or other colostrum SAA inducing agent to stimulate MAA production.

The MAA protein has been shown to stimulate the immune system and thus expression of MAA may be manipulated to increase or decrease MAA production to alleviate conditions associated with disease states particularly those associated with microbial infection. For example MAA upregulates MUC3, important immune factors in the intestine, and increased MAA will help fight diseases such as enteric colitis. Agonists of the promoter can be identified by the assays herein and used as pharmaceutical compositions to treat disorders associated with MAA over production.

The specificity of the promoter can also be used in transgenic protocols to direct expression of recombinant nucleotides in mammary tissues or more specifically in mammary epithelial cells. This includes expression constructs, vectors (replication and expression) and transformed recipient cells for large scale production of recombinant protein, creation of transgenic cows and the like.

Other features and advantages of the present invention will be better understood by reference to the drawings, detailed descriptions and examples that follow.

### BRIEF DESCRIPTION OF THE DRAWINGS

FIG. 1. FIG. 1 is the genomic bovine colostrum associated SAA sequence comprising the promoter of the invention as well as introns, and 3′ flanking region of the gene. (SEQ ID NO: 1). The TATA box is double underlined in the promoter region for bovine mammary-associated serum amyloid A 3 (M-SAA3, MAA). The additional three upstream nucleotides (single underlined) from the TATA box are also conserved in most “milk protein” TATA boxes. Putative consensus STAT5 DNA-binding sites at −1741 to −1749 and at −2184 to −2193 are italicized in bold type and denoted above the sequence. Other possible transcription factor DNA-binding sites are underlined and the corresponding transcription factor for this consensus sequence is identified above the sequence (MatInspector V2.2/TRANSFAC 4.0, Quandt et al, 1995). The transcriptional start site is underlined and indicated above the nucleotide with +1. The beginning and ending of the three introns are denoted with an arrow above these regions. The start and stop codons are underlined and indicated above the nucleotides. The encoded amino acids are denoted under the double-stranded DNA sequence. The presumed signal peptide cleavage site to remove the leader sequence predicted by the SignalP program (Version 1.1) (Nielsen et al., 1997) with 100% certainty is denoted by an inverted triangle. The polyadenylation signal (−6307 to −6312) is underlined and the probable site for cleavage and polyadenylation is indicated with a double arrow.

FIG. 2 is a graph showing the production of colostrum-SAA by the MAC T bovine epithelial cells over a periods of 13 days of stimulation with different levels of lipopolysaccharide (prolactin). Measurable quantities of colostrum-SAA could be detected by day 1 when the cells were stimulated with prolactin at 5 μg/ml.

FIG. 3 is a graph showing the production of colostrum-SAA by the MAC T bovine epithelial cells over a periods of 13 days of stimulation with different levels of lipopolysaccharide (prolactin). Measurable quantities of colostrum-SAA could be detected by day 1 when the cells were stimulated with LPS at 5 μg/ml.

FIG. 4A is a graph and Northern blot showing the enhanced Muc3 transcript levels by either the bovine or human N-terminal MAA peptide containing the TFLK motif. The % increase in Muc3 mRNA by human intestinal HT29 cells incubated with either the probiotic L. rhamnosus strain GG (LrGG) (P<0.05), the 10-mer with the scrambled TFLK motif at 50 μg/mL (Limited Scramble, LS) (P>0.05), the N-terminal bovine MAA 10-mer at 50 μg/mL (Bov) (P<0.05), and the N-terminal human MAA 10-mer at 50 μg/mL (Hum) (P<0.05), relative to untreated cells (None) (P>0.05), is shown in the graph. Amino acid sequences for the 10-mer peptides are detailed in Table 1 referred to on page 37. A representative northern blot hybridized with the Muc3-specific cDNA probe (upper panel of insert) and a probe complementary to 18S rRNA (lower panel of insert) is displayed in the insert to the right of the graph. Results are shown as means ±SEM of 3 separate experiments for each peptide or the bacterial probiotic. There was a 3.5- to 4.3-fold increase in Muc3 mRNA levels by HT29 cells incubated with the N-terminal MAA peptides containing the TFLK motif or bacterial probiotic, when compared to the untreated cells (ANOVA, P<0.05).

FIG. 4B is a graph showing the comparative inhibition of EPEC adherence to HT29 human intestinal cells grown in a glucose-free galactose-containing cell culture medium to enhance MUC3 expression. Each well received either 10⁹ CFU of the probiotic L. rhamnosus strain GG (LrGG), a single dose (1×) of the 10-mer peptide at 50 μg/mL (LS, Bov50, or Hum50) or at 250 μg/mL (Bov250 or Hum250), or two separate doses (2×) at 1 h intervals of the 10-mer peptide at 250 μg/mL. Samples were incubated at 37° C. with the HT29 cells for either a total of 1 h (single dose, 1×) or 2 h (two doses, 2×) prior to the addition of 10⁶ CFU/well of EPEC. After a 3 h incubation, EPEC adherent to the HT29 cells were quantified by determining CFU/well. Results are expressed as means ±SEM of 2-6 independent experiments run in triplicate. Amino acid sequences for the 10-mer peptides are detailed in Table 1 referred to on page 37. Both the bacterial probiotic (LrGG) (P<0.03) and the MAA-based 10-mer peptides containing the TFLK motif (Bov50, Bov250, Hum50, or Hum250) (P<0.03) significantly decreased adherence of EPEC, relative to untreated cells (None) (P>0.05).

### DETAILED DESCRIPTION OF THE INVENTION

I. Definitions

Various terms relating to the compositions and methods of the present invention are used herein above and also throughout the specification and claims.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “colostrum associated serum amyloid A”, “colostrum associated SAA” and/or “colostrum SAA” or “MAA” are used interchangeably and include but are not limited to the sequences disclosed herein, such as colostrum SAA3, their conservatively modified variants, regardless of source and any other variants which retain the biological properties of the colostrum SAA as demonstrated by the assays disclosed herein.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention. With respect to noncoding nucleic acid sequences conservatively modified, variants refers to these variants which retain the promoter activity of the sequence as determined by the assays disclosed herein.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   See also, Creighton (1984) Proteins W. H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al Nucl. Acids Res. 17:477-498 (1989)). Thus, the maize preferred codon for a particular amino acid may be derived from known gene sequences from maize. Maize codon usage for 28 genes from maize plants are listed in Table 4 of Murray et al., supra.

As used herein “full-length sequence” in reference to a specified polynucleotide or its encoded protein means having the entire amino acid sequence of, a native (non-synthetic), endogenous, biologically active form of the specified protein. Methods to determine whether a sequence is full-length are well known in the art including such exemplary techniques as northern or western blots, primer extensions, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

With respect to proteins or peptides, the term “isolated protein (or peptide)” or “isolated and purified protein (or peptide)” is sometimes used herein. This term may refer to a protein that has been sufficiently separated from other proteins with which it would naturally be associated, so as to exist in “substantially pure” form. Alternatively, this term may refer to a protein produced by expression of an isolated nucleic acid molecule.

With reference to nucleic acid molecules, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (this term is defined below).

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid maybe incorporated into the genome of the cell (e.g., chromosome, plasmid, plastid or mitochondrial DNA), converted into an autonomous replicon, or transiently expressed (e.g., transfected mRNA).

As used herein, “localized within the chromosomal region defined by and including” with respect to particular markers includes reference to a contiguous length of a chromosome delimited by and including the stated markers.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, phosphorylation, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention. With respect to a protein, the term “N-terminal region” shall include approximately 50 amino acids adjacent to the amino terminal end of a protein.

As used herein “TFLK motif” shall include any formulation whether by amino acids or otherwise that would maintain the structural integrity and biological activity of the TFLK active site of colostrum SAA.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and may be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl 1% SDS at 37° C., and awash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology-13 Hybridization with Nucleic Acids Probes, Part I, Chapter 2, Ausubel, et al., Eds., Greene Publishing and Wiley-Interscience, New York (1995).

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

Nucleic acid sequences and amino acid sequences can be compared using computer programs that align the similar sequences of the nucleic or amino acids thus define the differences. The BLAST programs (NCBI) and parameters used therein are used by many practitioners to align amino acid sequence fragments. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by Best-Fit program may also be used to compare sequence identity and similarity.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

With respect to oligonucleotides or other single-stranded nucleic acid molecules, the term “specifically hybridizing” refers to the association between two single-stranded nucleic acid molecules of sufficiently complementary sequence to permit such hybridization under predetermined conditions generally used in the art i.e., conditions of stringency (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S 1), as well as protein binding domains (consensus sequences) responsible for the binding of RNA polymerase. Thus as used herein the term promoter shall include any portion of genomic DNA disclosed herein which is capable of initiating expression of operably linked sequences at levels detectable above background.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In procaryotes, yeast, and mammalian cells for example, the transforming DNA may be maintained on an episomal element such as a plasmid. With respect to eucaryotic cells, a stably transformed cell is one in which the transforming DNA has become integrated into a chromosome so that it is inherited by daughter cells through chromosome replication. This stability is demonstrated by the ability of the eucaryotic cell to establish cell lines or clones comprised of a population of daughter cells containing the transforming DNA.

II. Description

Serum amyloid A (SAA) is an acute phase protein which is produced in the liver and occurs at elevated levels in the serum of mammals as part of the inflammatory response related to tissue injury or infection. The mammary associated isoform of SAA occurs at highly elevated levels in colostrum. Elevated colostrum SAA in healthy cows returns to background levels found in milk within four days after calving. Colostrum SAA is produced locally (i.e., in mammary ductal epithelial cells), in colostrum and occurs independently of the blood concentration of acute phase SAA (A-SAA) (in samples of colostrum, whey and serum taken from five test cows, serum SAA was found to be in the range of 15 μg/ml, while in colostrum, SAA was elevated to levels in the average range of 300 μg/ml).

Colostrum SAA may act as vehicle for transport of lipids and immunoglobulins across the endothelial membranes of gut and/or vasculature in the newborn. It may be produced locally by the vascular endothelium after injury, and serve as a vehicle for transport of immunoglobulins into the intravascular space. Colostrum SAA is likely to have anti-microbial activity (either directly or indirectly) and to regulate the immune response in some manner. Colostrum SAA also may be involved in tissue remodeling by inducing enzymes involved in tissue repair and degradation, and by regulating production of protective mucins in mucosal tissue. Thus it is useful as an antimicrobial and the specificity of the promoter may be used to cause the production of this antimicrobial protein in animals exposed to stress, infection or other microbial related condition.

The colostrum-derived SAAs share a unique amino acid sequence in the amino-terminal (or N-terminal) end TFLK (TFLK motif). The TFLK motif is not found in any of the serum-derived SAAs from any mammal, but does share homology with SAA3 produced by rabbit synovial fibroblasts.

Also according to the invention the genomic sequence of bovine colostrum associated SAA has been determined including several intron regions, (3) and the 3′ flanking region as well as the promoter region have been purified and isolated. The discovery of this promoter leads to several pharmacological and/or transgenic protocols which take advantage of the regulation of MAA expression to aid in treatment of disease, boost immunity, produce recombinant proteins and the like.

The specificity of the colostrum associated amyloid A promoter and regulatory regions of the invention may be exploited in any of a number of recombinant nucleotide protocols to direct expression to specific cells and tissues. The mammary associated SAA gene is expressed in mammary ductal epithelial cells and thus its promoter will cause expression of operably linked sequences for these cells and other mammary tissues. Similarly the mammary SAA gene product is expressed in higher levels and secreted in colostrum and in smaller amounts in milk. It is also induced by prolactin and/or LPS. The temporal and spatial specificity of this promoter can be used when operably linked to other non naturally occurring nucleotide sequences to target expression of these alternate genes in a similar manner. Methods to identify promoters from other mammalian species for mammary associated SAA include techniques known in the art as well as provided herein.

In one aspect the invention has provided an isolated promoter polynucleotide from a mammary associated amyloid A gene. In a particularly preferred embodiment of the invention, the polynucleotide comprises a promoter region from the bovine mammary associated amyloid A gene comprising a sequence set out in SEQ ID NO: 1 or a variant thereof.

It is a further aspect of the invention to provide isolated promoter and nucleic acid molecules from the mammary associated amyloid A gene including, for example, polynucleotides derived from such molecules as unprocessed RNAs, ribosome RNAs, mRNAs, cDNAs, genomic DNAs, B- and Z-DNAs. Further embodiments of the invention include biologically, diagnostically, prophylactically, clinically, or therapeutically useful polynucleotides and variants thereof and compositions comprising the same.

Another aspect of the invention relates to isolated polynucleotides including, for example, polynucleotides closely related to a mammary associated amyloid A promoter having a polynucleotide sequence of SEQ ID NO: 1 and variants thereof.

Using the information provided herein such as the promoter polynucleotide sequence of SEQ ID NO: 1, other promoter polynucleotides of the invention may be obtained using standard cloning and screening methods such as those for cloning and sequencing chromosomal (genomic) DNA fragments as disclosed herein. For example, to obtain a polynucleotide sequence of the invention such as the polynucleotide sequence given as SEQ ID NO: 1, typically a library of clones of chromosomal DNA of a mammalian species or some other suitable host is probed with a radiolabeled oligonucleotide preferably a 7-mer or longer derived from a partial sequence. Clones carrying DNA identical to that of the probe can then be distinguished using stringent hybridization conditions. By sequencing the individual clones as identified by the hybridization with sequencing primers designed from the original polynucleotide sequence, it is possible to extend the polynucleotide sequence in both directions to determine a functional promoter region sequence or full-length gene sequence. Conveniently such sequencing is performed, for example, using denatured double-stranded DNA prepared from a plasmid clone. Suitable techniques for accomplishing this objective is described in Maniantis et al., Molecular Cloning: A Laboratory Manual. 2^{nd}Edition, Cold Spring Harbor Laboratory Press, Cold Spring Harbor, N.Y. (1989). Direct genomic DNA sequencing may also be performed to obtain a promoter sequence of expressively linked full-length sequence.

Another very important method that can be used to identify cell type specific promoters that allow identification of genes expressed in a single cell is enhancer detection (O'Kane, C., and Gehring, W. J. (1987), “Detection in situ of genomic regulatory elements in Drosophila”, Proc. Natl. Acad. Sci. USA, 84, 9123-9127). This method was first developed in Drosophila and rapidly adapted to mice and plants (Wilson, C., Pearson, R. K., Bellen, H. J., O'Kane, C. J., Grossniklaus, U., and Gehring, W. J. (1989), “P-element-mediated enhancer detection: an efficient method for isolating and characterizing developmentally regulated genes in Drosophila”, Genes & Dev., 3, 1301-1313; Skarnes, W. C. (1990), “Entrapment vectors: a new tool for mammalian genetics”, Biotechnology, 8, 10 827-831; Topping, J. F., Wei, W., and Lindsey, K. (1991), “Functional tagging of regulatory elements in the plant genome”, Development, 112, 1009-1019; Sundaresan, V., Springer, P. S., Volpe, T., Haward, S., Jones, J. D. G., Dean, C., Ma, H., and Martienssen, R. A., (1995), “Patterns of gene action in plant development revealed by enhancer trap and gene trap transposable elements”, Genes & Dev., 9, 1979-1810).

In a further aspect, the present invention provides for an isolated polynucleotide comprising or consisting of a polynucleotide sequence which has at least 70% identity, preferably at least 80% identity, more preferably at least 90% identity, yet more preferably at least 95% identity, even more preferably at least 97-99%, or exact identity to SEQ ID NO: 1 over the entire length of the sequence ID, or polynucleotides which hybridize under conditions of high stringency thereto and yet retain promoter activity as determined by the assays herein.

Promoter polynucleotides from a polynucleotide encoding a mammary associated amyloid A polypeptide, including homologues and orthologues from species other than bovine may be obtained by a process which comprises steps of screening an appropriate library under stringent hybridization conditions with a label there detectable probe consisting of or comprising of sequences of SEQ ID NO: 1 or a fragment thereof and isolating the promoter and/or full length gene and/or genomic clones containing the polynucleotide sequence, as previously described.

Preferred embodiments are polynucleotides that retain substantially the same 3 0 biological function or activity as the promoter region of SEQ ID NO: 1. The promoter polynucleotides of the invention may be employed for example as research reagents and materials for discovery and treatment of, and diagnostics for diseases, particularly animal diseases as further discussed herein relating to polynucleotide assays.

Assays of the invention may be performed by determining the effect of transcript level on cell phenotype. These assays will help to characterize, among other things, temporal relevance of transcription to phenotype. Promoter polynucleotides of the invention may be used for over-production of heterologous proteins in eucaryotes and prokaryotes. Polynucleotides of the invention may also be used to assess the binding of small molecules, substrates and ligands in, for example, cells, cell free preparations, chemical libraries, and natural product mixtures to identify agonists and antagonists of promoter activity. These substrates and ligands may be natural substrates and ligands, or may be structural or functional mimetics.

Accordingly, in a further aspect, the present invention provides for a method of screening compounds to identify those which stimulate or which inhibit the function of the polynucleotide of the invention as well as related polynucleotides, to regulate MAA production. In general agonists or antagonists may be employed for therapeutic and prophylactic purposes for diseases associated with MAA over or underproduction.

Compounds may be identified from a variety of sources, for example cells, cell free preparations, chemical libraries and natural product mixtures. Such agonists, antagonists, or inhibitors so identified may be natural or modified substrates, ligands, receptors, enzymes, etc., or may be structural or functional mamatics thereof. These screening methods may simply measure the binding of a candidate compound to the polynucleotide or to cells or membranes bearing the polynucleotide. Alternatively the screening method may involve competition with a labeled competitor. Further these screening methods may test whether the candidate compound results in a signal generated by activation or inhibition of the polynucleotide using detection systems appropriate to the cells comprising the polynucleotide. Inhibitors of activation are generally assayed in the presence of a known agonist and the effect on activation by the agonist by the presence of the candidate compound is observed. Constitutively active promoter polynucleotides and/or constitutively expressed polynucleotides may be employed in screening methods for inverse agonists or inhibitors in the absence of an agonist or inhibitor by testing whether the candidate compound results in inhibition of activation of the polynucleotide, as the case maybe.

Further the screening methods may simply comprise the steps in mixing a candidate compound with a solution containing polynucleotide of the invention to form a mixture measuring MAA promoter polynucleotide activity in the mixture and comparing the MAA promoter polynucleotide activity of the mixture to a standard.

The methods of screening may involve high through-put techniques. For example to screen for agonists or antagonists, a synthetic reaction mix, a cellular compartment, such as a membrane, cell envelope or cell wall, or a preparation of any thereof, comprising a MAA polynucleotide and a labeled substrate or ligand of such polynucleotide is incubated in the absence or presence of a candidate molecule that may be a MAA agonist or antagonist. The ability of the candidate molecule to agonize or antagonize the MAA polynucleotide is reflected in decreased binding of the labeled ligand or decreased production of the product from such substrate. Molecules that bind gratuitously (i.e., without inducing the effects of MAA polynucleotide) are most likely to be good antagonists. Molecules that bind well and, as the case may be, increase the rate of product production from substrate, increase signal transaction or increase chemical channel activity are agonists. Detection of the rate or level as the case may be production of the product from the substrate signal transection or chemical channel activity may be enhanced by using reporter system. Reporter systems that may be useful in this regard include but are not limited to colormetric, labeled substrate converted into product, a reporter gene that is responsive to changes in MAA polynucleotide activity and binding assays known in the art.

Polynucleotides of the invention may be used to identify promoter binding proteins, such as sigma factors, if any, for such polynucleotide, through standard binding techniques known in the art, for example, gel retardation assays. Other of these techniques include, but are not limited to, ligand binding and crosslinking assays in which the polynucleotide is labeled with a radioactive isotope (for instance, ³²P), chemically modified (for instance, biotinylated or fluorescent tagged), or fused to a polynucleotide sequence suitable for detection or purification, and incubated with a source of the putative binding compound or ligand (e.g., cells, cell membranes, cell supernatants, tissue extracts, bodily materials). Other methods include biophysical techniques such as surface plasmon resonance and spectroscopy. These screening methods may also be used to identify agonists and antagonists of the polynucleotide which compete with the binding of the polynucleotide to its ligand(s), if any. Standard methods for conducting such assays are well understood in the art.

The fluorescence polarization value for a fluorescently-tagged molecule depends on the rotational correlation time or tumbling rate. Protein-polynucleotide complexes, such as formed by MAA polynucleotide associating with polypeptide or other factor, labeled to comprise a fluorescently-labeled molecule will have higher polarization values than a fluorescently labeled monomeric polynucleotide. It is preferred that his method be used to characterize small molecules that disrupt polypeptide-polynucleotide complexes.

Fluorescence energy transfer may also be used to characterize small molecules that interfere with the formation of MAA polynucleotide-polypeptide dimers, trimers, tetramers or higher order structures, or structures formed by MAA polynucleotide and a polypeptide or polypeptides. MAA polynucleotide can be labeled with both a donor and acceptor fluorophore. Upon mixing of the two labeled species and excitation of the donor fluorophore, fluorescence energy transfer can be detected by observing fluorescence of the acceptor. Compounds that block dimerization will inhibit fluorescence energy transfer.

In other embodiments of the invention there are provided methods for identifying compounds which bind to or otherwise inject with and inhibit or activate an activity or expression of the polynucleotide of the invention comprising: contacting a polynucleotide of the invention with a compound to be screened under conditions to permit binding to or other interaction between the compound and the polynucleotide to assess the binding to or other iron with the compound, such binding or interaction preferably being associated with a second component capable of providing a detectable signal in response to the binding or interaction of the polynucleotide with the compound; and determining whether the compound binds to or otherwise interacts with and activates or inhibits an activity or expression of the polynucleotide by detecting the presence or absence of a signal generated from the binding or interaction of the compound with the polynucleotide.

Another example of an assay for MAA agonists or antagonists is a competitive assay that combines MAA and a potential agonist or antagonist with MAA-binding molecules, recombinant MAA binding molecules, natural substrates or ligands or substrate or ligand mimetics, under appropriate conditions for a competitive inhibition assay. MAA can be labeled, such as by radioactivity or a colorimetric compound, such that the number of MAA molecules bound to a binding molecule or converted to product can be determined accurately to assess the effectiveness of the potential antagonist or agonist.

Potential antagonists include, among others, small organic molecules, peptides, polypeptides that bind to a polynucleotide of the invention and thereby inhibit or extinguish its activity or expression. Potential antagonists also may be small organic molecules, a peptide, a polypeptide such as a closely related protein that binds the same sites on a binding molecule, such as a binding molecule, without inducing MAA promoter-induced activities, thereby preventing the action of MAA polynucleotides by excluding MAA polynucleotides from binding.

Potential antagonists include a small molecule that binds to and occupies the binding site of the polynucleotide thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56:560 (1991); “Oligodeoxynucleotides As Antisense Inhibitors Of Gene Expression,” CRC Press, Boca Raton, Fla. (1988), for a description of these molecules). Preferred potential antagonists include compounds related to and variants of MAA.

Other examples of polypeptide antagonists include oligonucleotides or proteins which are closely related to the ligands, substrates, receptors, enzymes, etc., as the case may be, of the polynucleotide, e.g., a fragment of the ligands, substrates, receptors, enzymes, etc.; or small molecules which bind to the polynucleotide of the present invention but do not elicit a response, so that the activity of the polynucleotide is prevented.

Certain of the polynucleotides of the invention are biomimetics, functional mimetics of the natural MAA polynucleotide. These functional mimetics may be used for, among other things, antagonizing the activity of MAA polynucleotide. Functional mimetics of the polynucleotides of the invention include but are not limited to truncated polynucleotides. For example, preferred functional mimetics include, a polynucleotide comprising the polynucleotide sequence set forth in SEQ ID NO: 1 lacking 5, 10, 20, 30, 40, 50, 60, 70 or 80 5′ and/or 3′ nucleotide residues, including fusion promoters comprising one or more of these truncated sequences. Polynucleotides of these functional mimetics may be used to drive the expression of expression cassettes and marker genes. It is preferred that these cassettes comprise 5′ and 3′ restriction sites to allow for a convenient means to ligate the cassettes together when desired. It is further preferred that these cassettes comprise gene expression signals known in the art or described elsewhere herein.

It will be readily appreciated by the skilled artisan that a polynucleotide of the present invention may also be used in a method for the structure-based design of an agonist, antagonist or inhibitor of the polynucleotide, by: (a) determining in the first instance the three-dimensional structure of the polynucleotide, or complexes thereof, (b) deducing the three-dimensional structure for the likely reactive site(s), binding site(s) or motif(s) of an agonist, antagonist or inhibitor; (c) synthesizing candidate compounds that are predicted to bind to or react with the deduced binding site(s), reactive site(s), and/or motif(s); and (d) testing whether the candidate compounds are indeed agonists, antagonists or inhibitors.

It will be further appreciated that this will normally be an iterative process, and this iterative process may be performed using automated and computer-controlled steps.

In a further aspect, the present invention provides methods of treating abnormal conditions such as, for instance, a disease, related to either an excess of, an under-expression of, an elevated activity of, or a decreased activity of MAA polynucleotide.

If the expression and/or activity of the polynucleotide is in excess, several approaches are available. One approach comprises administering to an individual in need thereof an inhibitor compound (antagonist) as herein described, optionally in combination with a pharmaceutically acceptable carrier, in an amount effective to inhibit the function and/or expression of the polynucleotide, such as, for example, by blocking the binding of ligands, substrates, receptors, ennres, etc., or by inhibiting a second signal, and thereby alleviating the abnormal condition. In still another approach, promoter activity can be inhibited using expression blocking techniques. This blocking is preferably targeted against transcription. An examples of a known technique of this sort involve the use of antisense sequences, either internally generated or separately administered (see, for example, O'Connor, J. Neurochem (1991) 56:560 in Oligodeoxynucleotides as Antisense Inhibitors of Gene Expression, CRC Press, Boca Raton, Fla. (1988)). Alternatively, oligonucleotides which form triple helices with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo. Thus promoter polynucleotides of the invention are useful for ascertaining the functionality or essentiality of the target gene (gene-of-interest) in a cell through expression blocking techniques. A method comprises “knocking-out” the transcription or expression of gene-of-interest by expressing an anti-sense sequence to the gene-of-interest under the transcriptional control of the promoter polynucleotides of the invention, particularly those contained in SEQ ID NO:1. In another embodiment, the method comprises, in a cell, (a) disabling (“knocking-out”) the gene-of-interest; (b) reintroducing, at the target gene locus, the gene-of-interest now under the operational control of the inducible promoter polynucleotides of the invention (particularly those contained in SEQ ID NO: 1); and (c) adding the induce: thereby providing information to the essentiality or functionality of the gene of interest.

In addition to assays of the invention and resultant pharmaceutical agents, the promoter polynucleotide may be used in transgenic protocols for production of recombinant proteins be they in vitro or in vivo MAA or other heterologous proteins.

The following description sets forth the general procedures involved in practicing the present invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1999) (hereinafter “Ausubel et al.” are used.

Association of DNA sequences provided by the invention with homologous or heterologous polynucleotide encoding DNA sequences, and allows in vivo and in vitro transcription from mRNA which, in turn, is susceptible to translation to provide recombinant proteins, and related poly- and oligo-peptides in large quantities. In a presently preferred nucleotide construct of the invention protein encoding DNA is operatively linked to the regulatory promoter polypeptide of the invention allowing for in vitro transcription and translation of the protein.

Incorporation of DNA sequences into prokaryotic and eucaryotic host cells by standard transformation and transfection processes, potentially involving suitable viral and circular DNA plasmid vectors, is also within the contemplation of the invention and is expected to provide useful proteins in quantities heretofore unavailable from natural sources. Use of mammalian host cells is expected to provide for such post-translational modifications (e.g. truncation, glycosylation, and tyrosine, serine, or threonine phosphorylation) as may be needed to confer optimal biological activity on recombinant expression products of the invention as more fully set forth hereinafter.

Most of the techniques which are used to transform cells, construct vectors, extract messenger RNA, prepare cDNA libraries, and the like are widely practiced in the art, and most practitioners are familiar with the standard resource materials which describe specific conditions and procedures. However, for convenience, the following paragraphs may serve as a guideline.

Nucleotide Constructs

Nucleotide constructs, according to the invention, may be created with the promoters of the invention operably linked to heterologous or even MAA encoding sequences to provide for the transcription and/or expression of these sequences in cells. Any nucleotide sequence, of which transcription and/or translation is desired in a host cell, may be used according to the invention. This may include homologous MAA sequences for a “additional dose” of MAA transcription as well as heterologous polypeptide encoding sequences, the production of which are desired in said cell. Examples of heterologous polypeptides desirable for production in mammary tissues includes, but is not limited to, lactoferrin, lysozyme, immunoglobulins, lactalbumin, bisalt stimulated lipase, human serum proteins such as albumin, immunoglobulins factor VIII, factor IX, protein C, etc. as well as industrial enzymes such as proteases, lipases, chitinases, and liganases from prokaryotic and eukaryotic sources. The recombinant DNA sequences for the constructs can include genomic or cDNA sequences encoding the recombinant polypeptide. These expression constructs may then be used in yet another embodiment to create transgenic nonhuman mammals which secrete these proteins in milk or other mammary tissues such as disclosed in U.S. Pat. No. 6,222,094 Hanson et al., “Transgenic Nonhuman Mammal Expressing the DNA Sequence Encoding Capacasin Mammary Gland and Milk”; U.S. Pat. No. 6,140,552, DeBoer et al., “Production of Recombinant Polypeptides by Bovine Species and Transgenic Methods”; U.S. Pat. No. 6,025,540, Hansson et al., “Transgenic Nonhuman Mammals Producing ECSOD Protein in Their Milk”; and U.S. Pat. No. 6,013,857, DeBoer, “Transgenic Bovines and Milk From Transgenic Bovines” the disclosures of which are incorporated herein by reference.

Hosts and Control Sequences

Both prokaryotic and eucaryotic systems may be used to express the nucleotide constructs of the invention; prokaryotic hosts are, of course, the most convenient for cloning procedures. Prokaryotes most frequently are represented by various strains of E. coli; however, other microbial strains may also be used. Plasmid vectors which contain replication sites, selectable markers and control sequences derived from a species compatible with the host are often used in addition to the promoter of the invention; for example, E. coli is typically transformed using derivatives of pBR322, a plasmid derived from an E. coli species by Bolivar, et al, Gene (1977) 2:95. pBR322 contains genes for ampicillin and tetracycline resistance, and thus provides multiple selectable markers which can be either retained or destroyed in constructing the desired vector. Commonly used prokaryotic control sequences which are defined herein to include promoters for transcription initiation, optionally with an operator, along with ribosome binding site sequences, include such commonly used promoters as the beta-lactase (penicillinase) and lactose (lac) promoter systems (Chang, et al, Nature (1977) 198:1056) and the tryptophan (trp) promoter system (Goeddel, et al, Nucleic Acids Res (1980) 8:4057) and the lambda derived P_{L} promoter and N-gene ribosome binding site (Shimatake, et al, Nature (1981) 292:128).

In addition to bacteria, eucaryotic microbes, such as yeast, may also be used as hosts. Laboratory strains of Saccharomyces cerevisiae, Baker's yeast, are most used although a number of other strains or species are commonly available. Vectors employing, for example, the 2μ origin of replication of Broach, J. R., Meth Enz (1983) 101:307, or other yeast compatible origins of replication (see, for example, Stinchcomb, et al, Nature (1979) 282:39, Tschumper, G., et al, Gene (1980) 10:157 and Clarke, L, et al, Meth Enx (1983) 101:300) maybe used. Control sequences for yeast vectors include promoters for the synthesis of glycolytic enzymes (Hess, et al, J Adv Enzyme Reg (1968) 7:149; Holland, et al, Biochemistry (1978) 17:4900). Additional promoters known in the art include the promoter for 3-phosphoglycerate kinase (Hitzeman, et al J Biol Chem (1980) 255:2073). Other promoters, which have the additional advantage of transcription controlled by growth conditions and/or genetic background are the promoter regions for alcohol dehydrogenase 2, isocytochrome C, acid phosphatase, degradative enzymes associated with nitrogen metabolism, the alpha factor system and enzymes responsible for maltose and galactose utilization. It is also believed terminator sequences are desirable at the 3′ end of the coding sequences. Such terminators are found in the 3′ untranslated region following the coding sequences in yeast-derived genes.

The colostrum SAA promoters can be used as an inducible promoters to cause the production of operably linked sequences in response to prolactin and/or LPS or can be used to facilitate temporal and spatial expression of linked gene sequences to the mammary tissue and excreted in milk of the transgenic animal. Any gene sequence capable of being expressed in a host cell may be operably linked to the promoter of the invention and used in this manner. For example bovine serum albumin could be operably linked to the colostrum SAA of the invention causing transgenic BSA to be produced in the colostrum of said animal.

It is also, of course, possible to express genes encoding polypeptides in eucaryotic host cell cultures derived from multicellular organisms. See, for example, Axel, et al, U.S. Pat. No. 4,399,216. These systems have the additional advantage of the ability to splice out introns and thus can be used directly to express genomic fragments. Useful host cell lines include bovine epithelial cells, VERO and HeLa cells, and Chinese hamster ovary (CHO) cells. Expression vectors for such cells ordinarily include promoters and control sequences compatible with mammalian cells such as the promoter of the invention in combination with, for example, the commonly used early and late promoters from Simian Virus 40 (SV 40) (Fiers, et al, Nature (1978) 273:113), or other viral promoters such as those derived from polyoma, Adenovirus 2, bovine papilloma virus, or avian sarcoma viruses. The controllable promoter, hMT1I (Karin, M., et al, Nature (1982) 299:797-802) may also be used. General aspects of mammalian cell host system transformations have been described by Axel (supra). It now appears, also that “enhancer” regions are important in optimizing expression; these are, generally, sequences found upstream or downstream of the promoter region in non-coding DNA regions. Origins of replication may be obtained, if needed, from viral sources. However, integration into the chromosome is a common mechanism for DNA replication in eucaryotes.

Transformations

Depending on the host cell used, transformation is done using standard techniques appropriate to such cells. The calcium treatment employing calcium chloride, as described by Cohen, S. N., Proc Natl Acad Sci (USA) 1972) 69:2110, or the rbC12 method described in Maniatis, et al, Molecular Cloning: A Laboratory Manual (1982) Cold Spring Harbor Press, p. 254 and Hanahan, D., J Mol Biol (1983) 166:557-580 may be used for prokaryotes or other cells which contain substantial cell wall barriers. For mammalian cells without such cell walls, the calcium phosphate precipitation method of Graham and van der Eb, Virology (1978) 52:546, optionally as modified by Wigler, M., et al, Cell (1979) 16:777-785 may be used. Transformations into yeast may be carried out according to the method of Beggs, J. D. Nature (1978)275:104-109 or of Hinnen, A., et al, Proc Natl Acad Sci (USA) (1978) 75:1929.

Vector Construction

Construction of suitable vectors containing the desired coding and control sequences employs standard ligation and restriction techniques which are well understood in the art. Isolated plasmids, DNA sequences, or synthesized oligonucleotides are cleaved, tailored, and relegated in the form desired.

The DNA sequences which form the vectors are available from a number of sources. Backbone vectors and control systems are generally found on available “host” vectors which are used for the bulk of the sequences in construction. Typical sequences have been set forth above. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleoside derivatives. The entire sequence for genes or cDNA's of sizable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, for example, Edge, M. D., Nature (1981) 292:756; Nambair, K. P., et al, Science (1984) 223:1299; Jay, Ernest, J Biol Chem (1984) 259:6311.

Synthetic oligonucleotides are prepared by either the phosphotriester method as described by Edge, et al, Nature (supra) and Duckworth, et al, Nucleic Acids Res (1981) 9:1691 or the phosphoramidite method as described by Beaucage, S. L., and Caruthers, M. H., Tet Letts (1981) 22:1859 and Matteucci, M. D., and Caruthers, M. H., J Am Chem Soc (1981) 103:3185 and can be prepared using commercially available automated oligonucleotide synthesizers. Kinasing of single strands prior to annealing or for labeling is achieved using an excess, e.g., approximately 10 units of polynucleotide kinase to 1 nmole substrate in the presence of 50 mM Tris, pH 7.6, 10 mM MgCl₂, 5 mM dithiothreitol, 1-2 mM ATP, 1.7 pmoles γ32P-ATP (2.9 mCi/mmole), 0.1 mM spermidine, 0.1 mM EDTA.

Once the components of the desired vectors are thus available, they can be excised and ligated using standard restriction and ligation procedures.

Site specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. In general, about 1 μg of plasmid or DNA sequence is cleaved by one unit of enzyme in about 20 μl of buffer solution; in the examples herein, typically, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods in Enzymology (1980) 65:499-560.

Restriction cleaved fragments may be blunt ended by treating with the large fragment of E. coli DNA polymerase I (Klenow) in the presence of the four deoxynucleotide triphosphates (dNTPs) using incubation times of about 15 to 25 min at 20° to 25° C. in 50 mM Tris pH 7.6, 50 mM NaCl, 6 mM MgC12, 6 mM DTT and 0.1-1.0 mM dNTPs. The Klenow fragment fills in at 5′ single-stranded overhangs but chews back protruding 3′ single strands, even though the four dNTPs are present. If desired, selective repair can be performed by supplying only one of the, or selected, dNTPs within the limitations dictated by the nature of the overhang. After treatment with Klenow, the mixture is extracted with phenol/chloroform and ethanol precipitated. Treatment under appropriate conditions with S1 nuclease or BAL-31 results in hydrolysis of any single-3 0 stranded portion.

Ligations are performed in 15-50 μl volumes under the following standard conditions and temperatures: for example, 20 mM Tris-Cl pH 7.5, 10 MM MgCl2, 10 mM DTT, 33 μg/ml BSA, 10 mM-50 mM NaCl, and either 40 μM ATP, 0.01-0.02 (Weiss) units T4 DNA ligase at 0 C (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 nM total end concentration). Intermolecular blunt end ligations are performed at 1 μM total ends concentration.

In vector construction employing “vector fragments”, the vector fragment is commonly treated with bacterial alkaline phosphatase (BAP) or calf intestinal alkaline phosphatase (CIP) in order to remove the 5′ phosphate and prevent self-ligation of the vector. Digestions are conducted at pH 8 in approximately 10 mM Tris-HCl, 1 mM EDTA using about 1 unit of BAP or CIP per μg of vector at 60° for about one hour. In order to recover the nucleic acid fragments, the preparation is extracted with phenol/chloroform and ethanol precipitated. Alternatively, re-ligation can be prevented in vectors which have been double digested by additional restriction enzyme digestion and/or separation of the unwanted fragments.

For portions of vectors derived from cDNA or genomic DNA which require sequence modifications, site specific primer directed mutagenesis may be used (Zoller, M. J., and Smith, M. Nucleic Acids Res (1982) 10:6487-6500 and Adelman, J. P., et al, DNA (1983) 2:183-193). This is conducted using a primer synthetic oligonucleotide complementary to a single stranded phage DNA to be mutagenized except for limited mismatching, representing the desired mutation. Briefly, the synthetic oligonucleotide is used as a primer to direct synthesis of a strand complementary to the phage, and the resulting partially or fully double-stranded DNA is transformed into a phage-supporting host bacterium. Cultures of the transformed bacteria are plated in top agar, permitting plaque formation from single cells which harbor the phage.

Theoretically, 50% of the new plaques will contain the phage having, as a single strand, the mutated form; 50% will have the original sequence. The resulting plaques are washed after hybridization with kinased synthetic primer at a wash temperature which permits binding of an exact match, but at which the mismatches with the original strand are sufficient to prevent binding. Plaques which hybridize with the probe are then picked, cultured, and the DNA recovered.

Verification of Construction

Correct ligations for plasmid construction can be confirmed by first transforming E. coli strain MC1061 obtained from Dr. M. Casadaban (Casadaban, M., et al, J Mol Biol (1980) 138:179-207) or other suitable host with the ligation mixture. Successful transformants are selected by ampicillin, tetracycline or other antibiotic resistance by using other markers depending on the mode of plasmid construction, as is understood in the art. Plasmids from the transformants are then prepared according to the method of Clewell, D. B., et al, Proc Natl Acad Sci (USA) (1969) 62:1159, optionally following chloramphenicol amplification (Clewell, D. B., J Bacteriol (1972) 110:667). Several mini DNA preps are commonly used, e.g., Holmes, D. S., et al, Anal Biochem Acids Res (1979) 7:1513-1523. The isolated DNA is analyzed by restriction and/or sequenced by the dideoxy nucleotide method of Sanger, F., et al, Proc Natl Acad Sci (USA) (1977) 74:5463 as further described by Messing, et al, Nucleic Acids Res (1981) 9:309, or by the method of Maxam, et al, Methods in Enzymology (1980) 65:499.

Hosts Exemplified

Host strains used in cloning and prokaryotic expression herein are as follows:

For cloning and sequencing, and for expression of construction under control of most bacterial promoters, E. coli strains such as MC1061, DH1, RR1, C600hfl, K803, HB101, JA221, and JM101 can be used.

Pharmaceutical Preparations

According to the invention Applicant has discovered that the colostrum associated SAA stimulates mucin production in the intestine and thus the promoter of the colostrum SAA may be used to increase or decrease mucin production; and agonist or antagonist compounds identified by the assays of the invention may be used to treat diseases associated with mucin production. This is significant as mucins have been shown to have a key role in the prevention and treatment of intestinal infections and many probiotics act through inducing mucin production. See Mack et al, “Probiotics inhibit enteropathogenic Escherechia coli adherence in vitro by inducing intestinal mucin gene expression”, 1999, Am J Physiol, 4 Part 1 G941-950, the disclosure of which is incorporated herein by reference. Thus the invention also includes pharmaceutical preparations for animals involving agonists and antagonists of the colostrum associated SAA promoter. Those skilled in the medical arts will readily appreciate that the doses and schedules of pharmaceutical composition will vary depending on the age, health, sex, size and weight of the animal rather than administration, etc. These parameters can be determined for each system by well-established procedures and analysis e.g., in phase I, II and III clinical trials.

For administration, the colostrum associated SAA agonists and antagonists can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

In general, in addition to the active compounds, the pharmaceutical compositions of this invention may contain suitable excipients and auxiliaries which facilitate processing of the active compounds into preparations which can be used pharmaceutically. Oral dosage forms encompass tablets, dragees, and capsules. Preparations which can be administered rectally include suppositories. Other dosage forms include suitable solutions for administration parenterally or orally, and compositions which can be administered buccally or sublingually.

The pharmaceutical preparations of the present invention are manufactured in a manner which is itself well known in the art. For example the pharmaceutical preparations may be made by means of conventional mixing, granulating, dragee-making, dissolving, lyophilizing processes. The processes to be used will depend ultimately on the physical properties of the active ingredient used.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added. Possible pharmaceutical preparations which can be used rectally include, for example, suppositories, which consist of a combination of the active compounds with the suppository base. Suitable suppository bases are, for example, natural or synthetic triglycerides, paraffinhydrocarbons, polyethylene glycols, or higher alkanols. In addition, it is also possible to use gelatin rectal capsules which consist of a combination of the active compounds with a base. Possible base material include for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers.

In addition to administration with conventional carriers, active ingredients may be administered by a variety of specialized delivery drug techniques which are known to those of skill in the art.

As used herein the term “an effective amount” shall mean an amount of colostrum associated SAA antagonist or agonist sufficient to increase or decrease MAA production so that the desired and measurable biologic effect associated with MAA is achieved.

The significance of mucins in intestinal infections lies in their ability to prevent the events necessary for infectious organisms to cause disease.

Mucins also inhibit the adherence of bacteria to the epithelial cells of the intestinal tract. Binding of bacteria to the lining cells of the gut is the first step in invasion, toxin delivery and development of diarrheal disease. If binding of the enteric pathogens is inhibited then disease does not develop. Thus the stimulation of the promoter nucleotide sequence enables mucin upregulation.

Mucins have also been shown to inhibit replication of viruses.

This demonstrates pharmaceutical applications of MAA promoter agonists and antagonists for numerous enteric pathologies. For example the prevention of traveler's diarrhea. Many infectious organisms are geographical in nature and travelers outside of their own areas have usually not been previously exposed to these organisms, thus have not developed immunity to them. Many people will take antibiotics before traveling, but some antibiotics have deleterious side effects causing organisms to resist to many antibiotics.

Another other potential use would be to prevent dysentery and other infectious diseases particularly for the military. Vaccine development is proving to be problematic. For example, failure of military recruits to take vaccination (anthrax vaccine) and disease caused by vaccinations leading to the removal from the market of the vaccine (rotavirus vaccine). Colostrum associated SAA is to be a rapid, safe and effective means to reduce or prevent intestinal-related infections.

Another example includes prevention or treatment of infant diarrhea. Breast fed infants have far fewer infections than formula fed infants. Since colostrum is a natural substance beneficial for infants and colostrum associated SAA is a component of colostrum, it will be an invaluable natural addition to formula. Such formulas are commonly commercially available such as Infamel™, Similac™, Carnation Good Start™, and Gerber™. Probiotics have been shown to reduce severity and shorten the recovery time for viral-caused diarrhea. Thus, another use for colostrum associated SAA would be for children with this condition, which would also have an economic impact by reducing hospital stays and costs.

Yet another example includes the prevention or treatment of necrotizing enterocolitis (NEC). This is a serious complication that occurs in premature infants. With the various reproduction techniques that are being used there has been a significant increase in the number of premature infants. Therapy for NEC has remained the same for the last few decades. Since bacteria in the gut of the premature infant have a major role in the development of NEC, therapy for this condition consists of keeping the infant from feeding, giving strong antibiotics and hoping that the bowel does not perforate.

Prevention of diarrhea caused by Rotovirus and other virus infections in the intestine. This is a worldwide problem of high magnitude. Most infected are infants 4 to 24 months of age and accounts for 80,000 hospitalizations per year in the USA with life threatening rotovirus induced diarrhea. Children in day care centers are also highly susceptible for rotovirus infection and also infectious diarrhea caused by Astroviruses which accounts for 10% of all pediatric diarrhea worldwide.

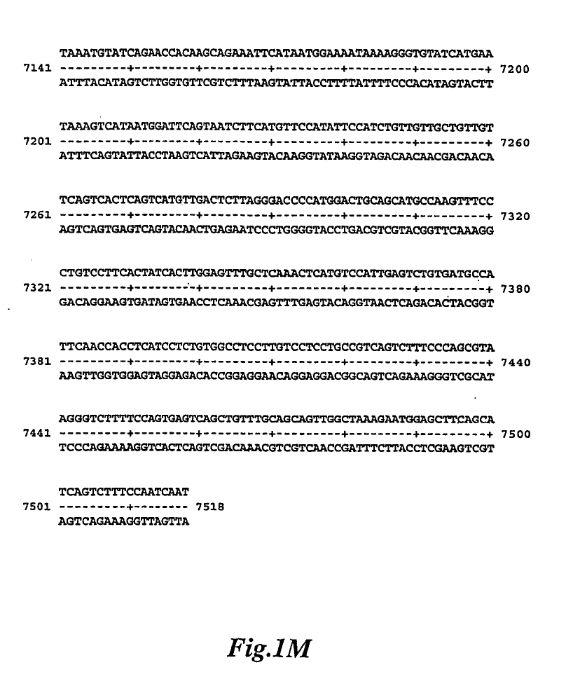

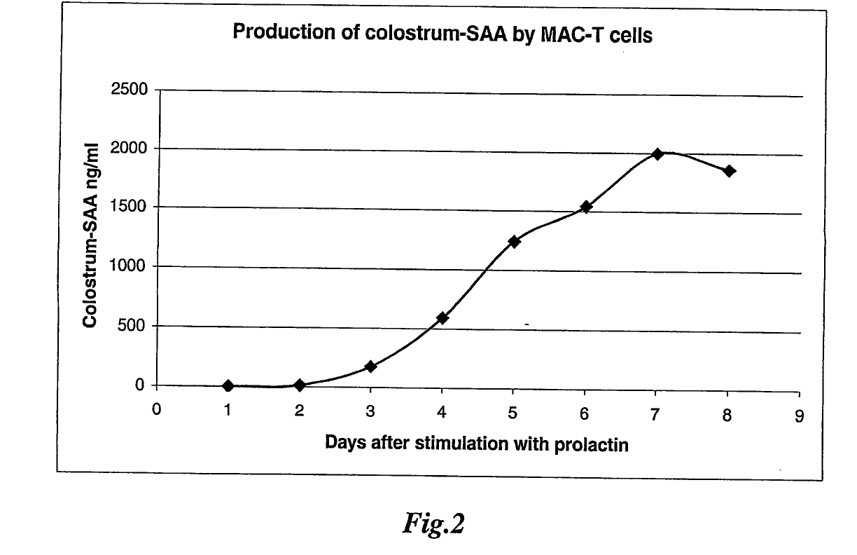

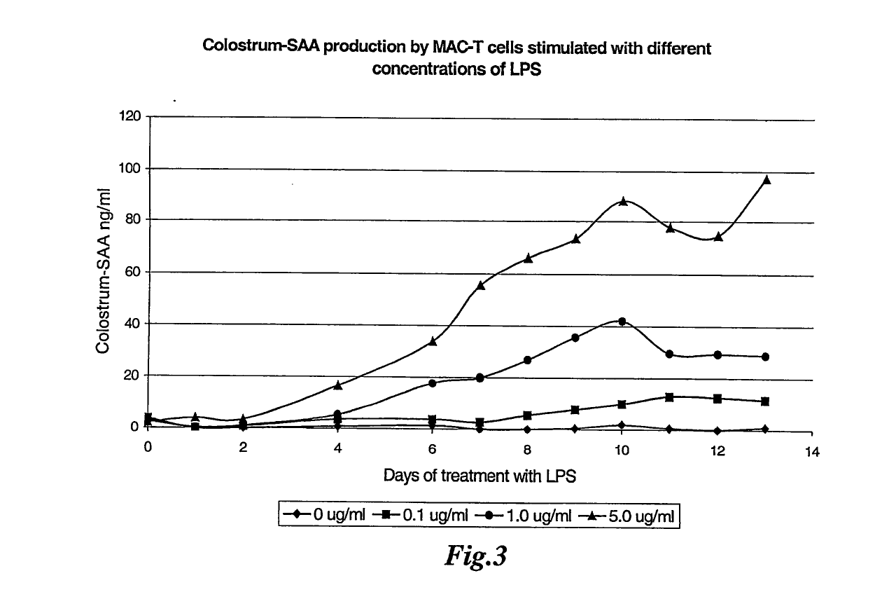

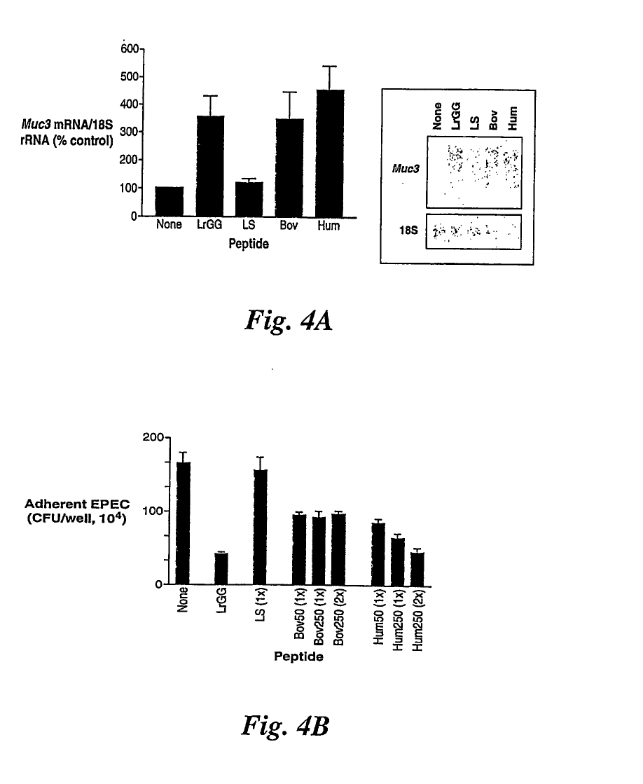

Another example includes the prevention of diarrhea in areas of outbreaks. E. coli 0157:H7 (see FIGS. 2A and 2B) outbreaks causing which can lead to deaths from hemolytic-uremic syndrome. We have shown that mucin production prevents E. coli from adhering to epithelial cells and thus could prevent this infection. In addition, treatment for prevention of the spread of intestinal infections in outbreaks of Salmonella and Shigella from food and water contamination sources are examples.

TABLE 1
Peptide Sequences

| | | Peptide | |
|---|---|---|---|
| No | Name | Sequence | Description |
| 1 | Limited | MWGLTKFEAG | Bovine MAA with |
| | Scramble (LS) | | TFLK motif scrambled |
| 2 | N-terminal Region | MWGTFLKEAG | 10-mer N-terminal |
| | (Bov) | | sequence of bovine MAA |
| | | | with TFLK motif |
| 3 | N-terminal Region | GWLTFLKAAG | 10-mer N-terminal |
| | (Hum) | | sequence of human MAA |
| | | | with TFLK motif |

Yet another example includes the treatment or prevention of urinary tract infections. The bladder epithelial cells are very similar to intestinal epithelial cells and are capable of producing mucins. Therefore prevention of urinary infections, including hospitalized patients with urinary catheters, would also be a use for the pharmaceutical compositions of the invention.

Yet another example includes the treatment or prevention of diarrhea in immunocomprimised patients. For example, about 40% of cancer patients acquire bacterial infections in the gut following radiation or chemotherapy which results in severe diarrhea. Clostridium difficle is most often isolated and identified as the offending bacterial agent. Increased mucin production could prevent diarrhea in these patients. Another application in immunocomprimised patient group is the prevention of intestinal infections and diarrhea in AIDS patients. The condition is primarily related to Cryptosporidia infections in the intestine.

In addition, prevention and treatment of diarrhea caused by Campylobacter infections in the intestine is another example. This condition is highly prevalent in the UK and USA with an incidence of over 100 per 100,000 population.

Yet another example includes veterinary medicine, for the prevention of infectious diarrhea in herd animals to allow for removal of antibiotics from the feed.

Yet another example includes the treatment or prevention of mastitis in cattle and other mammals. It is known that mucins are present in colostrums and milk and thought to be the key ingredient for the ability of milk to reduce intestinal infections in the neonate.

These mucins may also prevent bacterial adherence in the ducts of the mammary gland and prevent the initiation if mastitis. Increasing the production and consistency of mammary mucins by producing the their inducer, MAA would be a useful treatment application for the MAA promoter.

Although this disclosure includes upregulation of intestinal mucins, by administering a MAA promoter agonist, however epithelial cells lining other mucosal surfaces, (e.g. nasopharynx, bladder, mammary gland, etc.), also produce mucins. These mucins function to prevent infections analogous to intestinal mucins, and would also be effective targets for treatment according to the invention.

Other MAA associated diseases may also be treated by the MAA promoter agonists and antagonists of the invention. This may include microbial infections in general as MAA has been shown to regulate the immune system of the animal.

The following examples are provided to describe the invention in greater detail. They are intended to illustrate, not to limit, the invention.

### EXAMPLE 1

### Evaluation of SAA in Colostrum and Subsequent Serial Samplings of Milk

The purpose of this study was to evaluate colostrum and subsequent serial milk samplings to determine SAA content. Samples were obtained from Holstein dairy cows at the University of Nebraska—Lincoln Dairy Research Facility. Samples of colostrum were taken at calving, and subsequent milk samples were taken twice weekly for three weeks. Samples from all four udder quadrants were pooled. Results are shown in Table 2.

TABLE 2
SAA Levels in Colostrum and Milk Samples

| Cow ID | Sample Day | SAA ug/ml |
|---|---|---|
| 83 colostrum | Calving | 184.8 |
| 83 milk | +4 | 0.2 |
| 83 milk | +7 | 0.0 |
| 83 milk | +11 | 0.0 |
| 83 milk | +14 | 0.0 |
| 83 milk | +18 | 0.0 |
| 83 milk | +21 | 0.0 |
| 908 colostrum | Calving | 135.6 |
| 908 milk | +4 | 2.6 |
| 908 milk | +7 | 9.1 |
| 908 milk | +11 | 8.2 |
| 908 milk | +14 | 2.0 |
| 908 milk | +18 | 2.1 |
| 908 milk | +21 | 3.6 |
| 961 colostrum | Calving | 364.6 |
| 961 milk | +4 | 0.3 |
| 961 milk | +7 | 0.5 |
| 961 milk | +11 | 0.0 |
| 961 milk | +14 | 0.0 |
| 961 milk | +18 | 0.0 |
| 961 milk | +21 | 0.0 |

The results show that SAA is elevated in colostrum of normal animals but in very low levels or not detected in normal milk samples after colostrum has cleared.

### EXAMPLE 2

### Purification of SAA from Colostrum

The procedure set forth below can be used for purification of SAA from serum, plasma, milk or colostrum from any animal species. The procedure comprises two basic steps: the SAA is purified to approximately 20% purity by hydrophobic chromatography, then further purified to approximately 95% purity by SDS-PAGE and electro-blotting.

Approximately 30 ml of octyl sepharose CL-4B (Pharmacia #17-0790-01) was prepared by washing it with approximately 10 volumes (300 ml ) water to remove any traces of ethanol. This may be done by washing the gel in a sintered glass funnel (coarse, funnel volume 600 ml) or by adding the water to the gel in a beaker, and then allowing the gel to settle before pouring off the water and rewashing the gel.

The final washes (2×40 ml) of the gel were with a solution of 0.5M ammonium sulfate.

Prior to use, the colostrum was allowed to set at 4° C. to allow the lipid layer to separate from the aqueous layer, since the lipid portion seemed to interfere with the purification procedure. After the ammonium sulfate was poured off, 20 ml of the 4° C. refrigerated colostrum with elevated levels of SAA (preferably>100 μg/ml) was added to the gel (in the beaker). The suspension of colostrum and gel was swirled several times during the one hour incubation at room temperature so to allow the SAA to bind to the gel.

The gel was then poured into a 600 ml sintered glass funnel (coarse) and the non-bound fraction was collected. This non-bound fraction may be tested for SAA to determine the efficiency of binding.

The gel was washed with 5-times 50 ml 50 mM Tris, 10 mM NaCl buffer pH 7.6. The final wash should be clear.

The column was further washed with 2×50 ml of 30% isopropanol in Tris/NaCl. These washes were most thorough when a syringe with a 10 gauge needle was used to eject the isopropanol/buffer solution onto the gel. The gel was thoroughly mixed when this procedure was followed.

The SAA was eluted from the gel with a solution of 60% isopropanol in TRIS/NaCl. Generally this was done in four elutions of 10 ml each.

The eluates contained a variety of proteins, of which about 20% was SAA. In samples where the SAA was too dilute it was concentrated by evaporating die isopropanol in a centrifugal concentrator. (RC 1010, Jouan Inc.) For further purification for sequencing or amino acid analysis the proteins in the eluates were separated by SDS-PAGE and transferred to PVDF membrane by electro-blotting.

The band which was identified as SAA by SAA specific antibodies was then excised and sequenced.

RT-PCR Detection of colostrum associated SAA and not Acute Phase SAA mRNA Expression by Bovine Mammary Gland Epithelial Cells:

As previously described in detail, a 300 bp RT-PCR product was obtained from bovine mammary gland epithelial cells using the primer CDNA1-T14 for first strand synthesis from the mRNA present and then subsequent usage of the primers F1C and R3B for second strand synthesis and amplification. The figures show the nucleotide sequence obtained for this 300 bp RT-PCR product. This nucleotide sequence correlated with the peptide sequencing data obtained from the colostrum-associated and bovine mammary gland-associated SAA isoform.

The forward degenerate primer F2 (5′-GACATGTGGMGAGCCTACTCYGACATG-3′) and reverse degenerate primer R3B (previously described) were used in RT-PCR for amplification of A-SAA cDNA. The forward primer F2 corresponds to amino-terminal residues DMWRAYSDM in the acute phase SAA (A-SAA) protein (SWISS-PROT accession number P35541) and the reverse primer R3B corresponds to the carboxy-terminal residues SGKDPNHF in both the A-SAA protein and the colostrum associated SAA protein. Subsequent cloning and nucleotide sequencing of the resulting 267 bp CDNA sequences correlated with colostrum associated SAA cDNA, strongly suggesting that colostrum associated SAA and not A-SAA transcripts were present.

The restriction endonuclease XhoI site was found to be present in the cDNA sequence of bovine A-SAA (data not shown), but was not found in the cDNA sequence of bovine colostrum associated SAA. XhoI restriction endonuclease digestion of the 300 bp and 267 bp cDNA sequences previously described did not cleave either of these two RT-10 PCR products. This result additionally suggested that only colostrum associated SAA and not A-SAA mRNA was transcribed by bovine mammary gland epithelial cells.

To further verify that colostrum associated SAA and not A-SAA mRNA was expressed by the bovine mammary gland epithelial cells, the forward colostrum associated SAA-specific primer M3GW2 (previously described) and reverse CDNA1 primer (previously described) were again used in RT-PCR. In addition, the forward A-SAA-specific primer S3GW1 (5′-TAAGGGTACGACCAGTGGCCAGGGTCA-3′), corresponding to residues FKGTTSGQGQ in mature A-SAA) and the reverse CDNA1 primer (previously described) were used in RT-PCR. However, no RT-PCR product was observed using the forward A-SAA-specific primer and reverse CDNA1 primer, further confirming no expression or the low abundance of A-SAA mRNA expression by bovine mammary gland epithelial cells.

### EXAMPLE 3

### Colostrum-SAA Production by Bovine Mac-T Mammary Epithelial Cells Stimulated with Prolactin

Bovine MAC-T mammary gland epithelial cells were obtained from ATCC (CRL-10274) and cultured according to recommended conditions (Turner, J D and Huynh H. Immortalized bovine mammary epithelial cell line. U.S. Pat. No. 5,227,301 dated Jul. 13, 1993). MAC-T cells were cultured on Dulbecco's Modified Eagles Media (DMEM) supplemented with 10% Fetal Calf Serum (FCS), 5 μg/ml insulin, 1μg/ml hydrocortisone and fungizone. Cells were incubated at 37° C. with 5% CO₂. For colostrum-SAA production the cells were seeded onto type I Collagen coated plates. After 14 hours of incubation, the cells were washed twice with Dulbecco's phosphate buffered saline (DPBS) and incubated in media (DMEM, 5 μg/ml insulin, 1 μg/ml hydrocortisone and 2.5% FCS) supplemented with prolactin from sheep pituitary gland (5 μg/ml) for the stimulation of colostrum-SAA production. Approximately one half of the media was replaced daily with fresh prolactin supplemented media. Standard ELISA for the quantitation of SAA was used to assay aliquots of the growth media collected on the different days for the presence of colostrum-SAA.

Cells were kept in culture for 41 days in media supplemented with prolactin. Levels of colostrum-SAA production reached a maximum of almost 3000 ng/ml.

Purification of Colostrum Associated SAA from Cell Culture Fluid.

Colostrum-SAA was purified from cell culture fluid by affinity chromatography. Briefly, an affinity column was prepared by coupling a monoclonal antibody with specificity to SAA to cyanogen bromide activated sepharose 4B. Treating the column with 50 mM Tris, 0.1 NaCl, 0.2 M glycine pH 8 buffer blocked residual active groups on the gel. The column was then washed with 50 mM Tris, 0.1 M NaCl pH 7.2 buffer to remove any excess uncoupled protein. Approximately 50-ml culture fluid was passed over the column. The column was washed with a Tris-NaC 1 buffer to remove any nonbound proteins that were trapped in the column. The proteins were then eluted from the column with 0.1 M glycine-HCl pH 2.8. The fractions were neutralized immediately. All fractions were assayed by ELISA to determine which fraction contained the maximum amount of colostrum associated SAA and also by western blot to assess the total protein content of the fractions.

Determining the Amino Acid Sequence of the Purified Protein.

The fraction containing the greatest amount of the colostrum-SAA was subjected to 12% SDS-PAGE and electroblotted onto a PVDF membrane by a Mini Trans-Blot system (BioRad Laboratories). A section of one lane of the membrane was cut off and was stained with the monoclonal antibody to SAA to verify the presence of the colostrum-SAA. The remainder of the membrane was stained for five minutes in a solution of methanol:water (40:60) containing 0.5% (wt/vol) Coomassie Blue, and destained in a solution of methanol:water (50:50). The colostrum SAA proteins (identified by the monoclonal antibody stain) were excised from the membrane. The protein was deblocked using pfu pyroglutamate aminopeptidase (TaKaRa Biochemicals) followed by N-terminal sequencing using Edman degradation. Sequencing was performed on a Procise 491 made by PE-Biosystems through the University of Nebraska Medical Center's Protein Core Facility. The N-terminal sequence for colostrum-SAA was present, but not A-SAA.

Isoelectric Focusing (IEF) of SAA from Serum, Colostrum and Cell Culture Fluid.

The PROTEIN IEF Cell (BioRad) was used for the isoelectric focusing of the various SAA preparations. Ready Strip IPG Strips (BioRad Laboratories) with a pH range of 3-10 were used for the IEF. The second dimension (2-D) of the protein analysis was done by subjecting the IPG strips to 12% SDS-PAGE gel and electroblotting onto a PVDF membrane. The strips and blots were done in duplicate so that one of the blots could be stained with a protein stain, Coomassie Brilliant Blue, (CBB) and the other stained with the anti-SAA monoclonal antibody for the identification of the SAA protein isoforms. The samples also contained internal IEF standards (BioRad Laboratories) so that isoelectric point (pI) of each SAA isoforms could be determined. By comparing the antibody stained spots to the spots of the standards stained with CBB the apparent pI of the SAA isoforms could be determined. All of these procedures were done according to the protocol recommended by the manufacturer.

The proteins subjected to the IEF and 2-D analysis were either affinity purified as described for the cell culture fluid or, in the case of the serum only semi-purified by hydrophobic chromatography. SAA is a highly hydrophobic molecule and will bind readily to Octyl Sepharose beads and then under the appropriate conditions can be eluted off the matrix. Briefly, serum with an elevated SAA level was tumbled for one hour with an equal volume of Octyl Sepharose CL-4B gel to allow for hydrophobic binding of the proteins from the serum to the gel. The gel was washed with Tris-HCl buffer to remove any proteins that were just trapped within the matrix. The proteins bound to the gel were eluted by 60% isopropanol in Tris-NaCl buffer. These eluates contain a variety of proteins of which about 20% is SAA. An aliquot of this preparation or the affinity purified colostrum-SAA from the cell culture fluid or the colostrum was loaded onto the IPG strips and then standard procedures were followed for the IEF and the 2-D gel.

After analyzing the stained gels it was determined that the colostrum-SAA from both colostrum and MAC-T cell culture fluid had a pI of greater than 8 and was estimated to have an apparent pI of 9.4-9.6. The SAA from the serum contained three isoforms with apparent pI values of approximately 7.0, 5.8 and 5.5. There was no isoform in the serum that matched the pI of the colostrum-SAA.

### EXAMPLE 4

### Functional Roles of Colostrum-SAA

A remarkable feature of human physiology is that the mucosal epithelial cells that line the intestinal tract are in contact with a vast number of microbes and yet the incidence of infection and inflammatory complications is low. This suggests that local host protective mechanisms include highly effective, broad-spectrum, non-inflammatory antimicrobial defenses. Whereas the acquired immune system develops an effective response, it does so over a period of days or weeks and in infants the acquired immune system is immature and not fully functional. In contrast, the innate immune system of the intestinal tract is continual or immediately inducible to many potential pathogens introduced into the intestinal tract and brought into close proximity to the mucosal epithelial cells and functional at birth. Intestinal innate immunity includes first-line host-defense elements that range from simple inorganic molecules such as nitric oxide to natural killer cells. There are also a number of molecules produced by the epithelial cells that comprise an effector arm of the innate immune system. These include the relatively small antimicrobial peptides and more complex glycoprotein molecules such as mucins.

Mucins are secreted and cell-surface high-molecular-weight glycoproteins synthesized by epithelial cells of a number of organ systems including the intestinal tract. The strategic interpositioning between the intestinal lumen and the underlying mucosal epithelial cells of the intestinal tract has suggested that mucins have a number of important biological functions. In the intestinal tract, mucins protect against viral infections by inhibition of viral replication and enhancing viral clearance from the intestinal tract. Bacterial pathogens are prevented from adherence to intestinal epithelial cells. Adherence of enteropathogens is the crucial first step required for subsequent invasion, colonization or toxin delivery. Inhibition of adherence of enteric pathogens to intestinal epithelial cells by mucins could be by means of steric hindrance. Applicant's previous work and that of others has also shown that specific mucin-bacterial interactions could also be an important mechanism whereby mucins effect benefit for the host. However, regardless of the mechanism, prevention of mucosal infections is an important function of mucins.

Different mucin genes have been identified and to date, thirteen human mucin genes have been identified. However, MUC3 mucin is the predominant small intestinal mucin. It has previously been shown that the MUC3 mucins are effective in preventing the adherence of enteropathogenic Escherichia coli (EPEC) to intestinal epithelial cells. Applicant also showed that agents such as non-harmful bacteria that normally colonize the intestinal tract (i.e. probiotics) inhibit EPEC epithelial cell adherence and do so by the upregulation of intestinal mucin genes. It is also well known that breast-feeding infants are far less susceptible to infectious diarrhea than formula fed infants. There are a number of theories why this is so, but it is hypothesized that milk-associated amyloid (colostrum SAA) may be an important inducer of MUC3 gene expression. Applicant has evaluated MUC3 mRNA expression using an in vitro human cell culture assay system. In this system, intestinal cells incubated with the N-terminal peptide sequence of colostrum SAA have shown increased MUC3 mRNA expression as compared to control cells grown without the addition of colostrum SAA to the cell culture medium. To further explore this finding, Applicant has evaluated the functional specificity of the N-terminal peptide sequence of colostrum SAA by evaluation of MUC3 expression in the same in vitro assay using colostrum SAA N-terminal peptide sequences that have been randomly scrambled and a colostrum SAA peptide sequence downstream of the N-terminal sequence. If the expression of MUC3 is increased then intestinal cells grown in the presence of colostrum SAA should have a greater capacity to inhibit adherence of bacterial pathogens. This was studied (FIG. 2B) using EPEC in an in vitro assay system pre-incubated with colostrum SAA in the cell culture medium. Enteropathogenic E. coli are non-invasive, non-toxin producing pathogens that have been recognized as a significant cause of diarrhea in third world countries and in day care settings in developed countries. Future studies will evaluate the benefits of colostrum SAA in in vivo studies as well as characterized animal equivalent to human EPEC. Colostrum associated SAA provides a means to naturally upregulate the innate protective mechanisms of the human intestine and would provide a novel form of therapy to a common problem that occurs all too often in the third world leads to death of infants and in the developed world countries and leads to significant morbidity and cost. Thus, this is an effective, natural, non-drug/chemical therapy for all infectious diarrhea.

In order to address possible functional roles of colostrum-SAA, the Applicant synthesized, on a standard amino acid synthesizer, a 10 amino acid region of the molecule from bovine that represented the N-terminal portion of the mature protein containing the conserved TFLK. The peptide consisted of the following amino acids: MWGTFLKEAG (SEQ ID NO:30) (Named “N-Terminal”). Since it was anticipated that the TFLK amino acids would be the critical elements of the peptide, we also constructed a peptide in which those four amino acids were scrambled in their order MWGLTKFEAG (SEQ ID NO:28). (Named “Limited Scramble”). For controls we synthesized two peptides, one in which the amino acids in the entire N-Terminal peptide were arranged in random order GKFAWEGMTL (SEQ ID NO:29) (Named “Total Scramble”) and a 10 amino acid peptide in which the first 7 were from residues 62-69 of bovine SAADAAQRGPQQA (SEQ ID NO:30) (Named “C-Terminal”).

These four peptides were used in a cell culture assay designed to evaluate them for their properties of inducing mucin (MUC) mRNA production, either MUC3 or MUC2 according to the methods described by Mack et al. (Biochem. Biophys. Res. Commun. 199: 1012-1018, 1994 and Am J Physiol. Vol. 4, part 1, pg. G841-950, 1999).

N-Terminal Peptide Titration MUC3.

Intestinal epithelial cells, Mack et al. 1994, 1999, were exposed to the N-terminal 10 amino acid bovine colostrum-SAA peptide at various concentrations for 30 minutes incubation at 37° C. The cells were incubated an additional hour following replacement of the test medium with fresh medium without peptide and then the total cellular mRNA isolated and analyzed for MUC3 specific mRNA.

The N-terminal 10 amino acid, bovine Colostrum-SAA “N-terminal” peptide containing the TFLK motif stimulated the production of MUC3 mRNA up to 1½ times that of base line control levels (significance of P<0.002). The optimum concentration was 50 μg/ml medium.

ANOVA Table for MUC3/28S rRNA ration

| | | Sum of | Mean | F- | P- | | |
|---|---|---|---|---|---|---|---|
| | DF | Squares | Square | Value | Value | Lambda | Power |
| colostrum | 5 | 87931.344 | 17586.269 | 6.670 | .0002 | 33.349 | .996 |
| SAA | | | | | | | |
| Concen- | | | | | | | |
| tration | | | | | | | |
| Residual | 37 | 97557.446 | 2636.688 | | | | |

Means Table for MUC3/28S rRNA ration
Effect: colostrum SAA Concentration

| | Count | Mean | Std. Dev. | Std. Err. |
|---|---|---|---|---|
| 0 | 8 | 100.000 | 0.000 | 0.000 |
| 1 | 7 | 105.429 | 28.254 | 10.679 |
| 10 | 7 | 105.857 | 69.163 | 26.141 |
| 25 | 5 | 145.000 | 24.576 | 10.991 |
| 50 | 8 | 220.500 | 71.889 | 25.417 |
| 100 | 8 | 103.125 | 60.326 | 21.329 |

MUC3 Stimulation.

MUC3 stimulating activity of the N-terminal 10 amino acid bovine colostrum-SAA peptide was compared to the activity of the “Limited Scramble”, the “Total Scramble” and “C-Terminal peptides”. Optimum time and temperature of incubation and concentration of peptides was for 30 minutes at 37° C. at 50 μg/ml respectively. Data showed that the original N-Terminal peptide was the only peptide that stimulated MUC3 mRNA statistically significantly over the control values (P<0.008). Important note is that the lack of stimulation by the “Limited Scramble” peptide, in which only the novel TFLK sequence was rearranged strongly implies that this motif may be the key element in conferring biological activity and perhaps a rationale for it being conserved amongst species.

ANOVA Table for MUC2 mRNA/28S rRNA ratio

| | | Sum of | Mean | F- | P- | | |
|---|---|---|---|---|---|---|---|
| | DF | Squares | Square | Value | Value | Lambda | Power |
| colostrum | 4 | 6974.667 | 1743.667 | 5.081 | .0039 | 20.322 | .932 |
| SAA | | | | | | | |
| Peptides | | | | | | | |
| Residual | 25 | 8580.000 | 343.200 | | | | |

Mean Table for MUC2 mRNA/28s RNA ratio
Effect: colostrum SAA Peptides

| | Count | Mean | Std. Dev. | Std. Err. |
|---|---|---|---|---|
| N-terminal | 6 | 101.167 | 21.876 | 8.931 |
| Limited Scramble | 6 | 87.167 | 24.078 | 9.830 |
| Total Scramble | 6 | 65.833 | 20.605 | 8.412 |
| C-terminal | 6 | 67.500 | 15.268 | 6.233 |
| Control | 6 | 100.000 | 0.000 | 0.000 |

MUC2 Stimulation. To address whether the N-Terminal 10 amino acid bovine colostrum-SAA peptide would stimulate mRNA synthesis for MUC2 production, intestinal epithelial cells were cultured under conditions favoring MUC2 expression rather than MUC3. All peptides were used at concentrations and conditions that were previously optimized for MUC3 stimulation. None of the peptides stimulated the production of MUC2 mRNA. When comparing the N-Terminal 10 amino acid peptide that stimulated MUC3 to control baseline levels, the values were not significantly different. To show that the lack of MUC2 stimulation was not due to culture conditions, cells were exposed to the N-Terminal 10 amino acid bovine colostrum-SAA peptide at 2× and 5× optimum levels for MUC3 (100 and 500 μg/ml respectively). Additionally, conditions were changed to 2× the optimum MUC3 incubation time (1 hr). None of these changes resulted in MUC2 mRNA increase over control values. The evidence strongly indicates specificity of function in stimulating the production of a mucin produced mainly in the small intestine (MUC3) over one produced primarily in the large intestine (MUC2).

ANOVA Table for MUC3 mRNA/28S rRNA ratio

| | | Sum of | Mean | F- | P- | | |
|---|---|---|---|---|---|---|---|
| | DF | Squares | Square | Value | Value | Lambda | Power |
| colostrum | 4 | 25215.200 | 6303.800 | 4.387 | .0080 | 17.550 | .886 |
| SAA | | | | | | | |
| Peptides | | | | | | | |
| Residual | 25 | 35919.500 | 1436.780 | | | | |

Mean Table for MUC3 mRNA/28S rRNA ratio
Effect: colostrum SAA Peptides

| | Count | Mean | Std. Dev. | Std. Err. |
|---|---|---|---|---|
| N-terminal | 6 | 174.833 | 22.266 | 9.090 |
| Limited Scramble | 6 | 95.000 | 38.063 | 15.539 |
| Total Scramble | 6 | 109.333 | 56.874 | 23.219 |
| C-terminal | 6 | 111.333 | 44.774 | 18.279 |
| Control | 6 | 100.000 | 0.000 | 0.000 |

The present invention is not limited to the embodiments described above, but is capable of modification within the scope of the appended claims.

### EXAMPLE 5

### Colostrum-SAA Production by Bovine MAC-T Mammary Epithelial Cells after they are Stimulated with LPS

Stimulation of MAC-T mammary epithelial cells by Lipopolysaccharide (LPS)

Bovine MAC-T mammary gland epithelial cells were obtained from ATCC (CRL-10274) and cultured according to recommended conditions (Immortalized bovine mammary epithelial cell line. U.S. Pat. No. 5,227,301 dated Jul. 13, 1993). MAC-T cells were cultured on Dulbecco's Modified Eagles Media (DMEM) supplemented with 10% Fetal Calf Serum (FCB), 5μg/ml insulin, 1 μg/ml hydrocortisone and fungizone. Cells were incubated at 37° C with 5% CO₂. Typically, for colostrum-SAA production the cells were seeded at 2.5×10⁵ cells/cm² onto type I Collagen coated plates. After 14 hours of incubation, the cells were washed twice with Dulbecco's phosphate buffered saline (DPBS) and incubated with media (DMEM, 5μg/ml insulin, 1 μg/ml hydrocortisone and 2.5% FCS) supplemented with lipopolysaccharide from E. coli. The concentration of LPS added to the media for the stimulation of colostrum-SAA production ranged from 0 to 5 μg/ml. Essentially all of the media was replaced daily with fresh LPS supplemented media. Standard ELISA for the quantitation of SAA was used to assay aliquots of growth media collected daily for the presence of the colostrum-SAA.

For the production of colostrum-SAA, the cells could also be cultured on regular tissue culture plates (non-collagen coated). Cells were kept in culture for 40 days on these plates in media supplemented with LPS at 10 μg/ml with a daily replacement of the media. Levels of colostrum-SAA production reached a maximum of almost 30 μg/ml.

Aliquots of the culture fluid from the MAC-T cells collected at various times over the period of 27 days were analyzed for protein content. The proteins from these samples were separated by SDS-PAGE on a 10% gel and then silver stained. The protein profile changed over the period of 27 days. Although the individual proteins were not identified, changes in overall banding patterns could be observed, particularly between the molecular weight range of 15-25 kDa. Here, several proteins could be detected by day 3 but then were no longer detectable by day 18. The production of colostrum-SAA, as determined by ELISA, increased in concentration from 0.001 μg/ml on day 1 to approximately 25 μg/ml by day 27.

Purification of Colostrum-Associated SAA from Cell Culture Fluid.

Colostrum-SAA was purified from cell culture fluid by affinity chromatography. Briefly, an affinity column was prepared by coupling monoclonal antibody with specificity to SAA to cyanogen bromide activated sepharose 4B. Treating the column with 50-mM Tris, 0.1 M NaCl, 0.2-M glycine pH 8 buffer blocked residual active groups on the gel. The column was then washed with 50 mM Tris, 0.1 M NaCl pH 7.2 buffer to remove any excess uncoupled protein. Approximately 50-ml culture fluid was passed over the column. The column was then washed with the Tris-NaCl buffer to remove any non-bound proteins that were trapped in the column. A 0.1-M glycine-HCl pH 2.8 buffer was used to elute the proteins from the column. The fractions were neutralized immediately. All fractions were assayed by ELISA to determine which fractions contained the maximum amount of colostrum associated SAA.

Determining the Amino Acid Sequence of the Purified Protein.

The fraction containing the greatest amount of the colostrum-SAA was subjected to 12% SDS-PAGE and electroblotted onto a PVDF membrane by a Mini Trans-Blot system (BioRad Laboratories). A section of one lane of the membrane was cut off and was stained with the monoclonal antibody to SAA to verify the presence of the colostrum-SAA. The remainder of the membrane was stained for five minutes in a solution of methanol:water (40:60) containing 0.5% (wt/vol) Coomassie Blue, and destained in a solution of methanol:water (50:50). The colostrum SAA proteins (identified by the monoclonal antibody stain) were excised from the membrane. The protein was deblocked using pfu pyroglutamate aminopeptidase (TaKaRa Biochemicals) followed by N-terminal sequencing using Edman degradation. Sequencing was performed on a Procise 491 made by PE-Biosystems through the University of Nebraska Medical Center's Protein Core Facility. The N-terminal sequence for colostrum-SAA was present, but not A-SAA.

Isoelectric Focusing (IEF) of SAA from Colostrum and Cell Culture Fluid.

The PROTEIN IEF Cell (BioRad) was used for the isoelectric focusing of the MAA preparations. The samples of MAA from colostrum and MAC T-cell fluid had been prepared by affinity purification. Ready strip IPG Strips (BioRad Laboratories) with a pH range of 3-10 and 7-10 were used for the IEF. The second dimension (2-D) of the protein analysis was done by subjecting the IPG strips to 12% SDS-PAGE gel and electroblotting onto a PVDF membrane. The blots were stained with anti-MAA antibody for the identification of the MAA protein isoforms. By comparing the spots stained with the antibody on the blot of the 2D gel from the MAA purified from colostrum and the MAA from the culture fluid of MAC-T cells stimulated with LPS, the similarities of the isoforms could be determined. Each sample was first analyzed on a pH 3-10 IPG strip for the first dimension of separation and then a subsequent sample analyzed on a pH 7-10 IPG strip.

After analyzing the stained gels it was determined that the MAA purified from the culture fluid of MAC-T cells stimulated with LPS contains only one isoform of MAA with the pI of 9.4-9.6. This is identical to the pI of the MAA purified from colostrum and not to the isoforms associated with the acute phase response. LPS is a compound that normally elicits an inflammatory response. MAA that is produced by the MAC-T cells as a result of LPS stimulation is the same as the colostrum MAA produced as a result of hormonal (prolactin) stimulation of the MAC-T cells and also the same isoform that is present in colostrum.

### EXAMPLE 6

### Bovine Colostrum Serum Amyloid A 3 Genomic Sequence Cloning of Sequence Identification No. 1:

The cDNA and amino acid sequence for bovine M-SAA3 (MAA) was obtained as previously described herein and has been deposited in the GenBank database under Accession No. AF335552. The nucleotide sequence of the introns, promoter, and 3′ flanking regions for the bovine M-SAA3 gene were determined by PCR amplification and genomic walking procedures (Universal Genome Walker Kit; Clonetech). The primary and nested secondary M-SAA3 gene-specific primers used in the genomic walking procedures were designed according to the manufacturer's recommendations and were initially complimentary to either the 5′ or 3′ region of the bovine M-SAA3 cDNA previously described. Primary PCRs were carried out in 25 μL volumes containing 200 μM of adapter primer (AP 1), 200 μM deoxynucleoside triphosphates, 1.1 mM magnesium acetate, 15 mM potassium acetate, 40 mM Tris-HCl (pH 9.3), 1 U of rTth DNA polymerase XL (Perlin-Elmer), and approximately 25 ng of adapter-ligated bovine genomic DNA digested with either StuI, ScaI, Hind III, or SspI. The thermal cycling parameters used were 7 cycles for 15 seconds at 94° C. and 3 minutes at 72° C., 37 cycles for 15 seconds at 94° C. and 3 minutes at 67° C., and then 1 cycle for 4 minutes at 67° C. Secondary PCRs were carried out in 50 μL volumes containing 1μL of a 1:50 dilution of the appropriate primary PCR mixture, adapter primerAP2, and either a forward or reverse nested M-SAA3 gene-specific primer. The other reaction components and thermal cycling parameters were the same as those used for primary PCR.

Nucleotide Sequencing and Computer Analysis of Genomic Sequence:

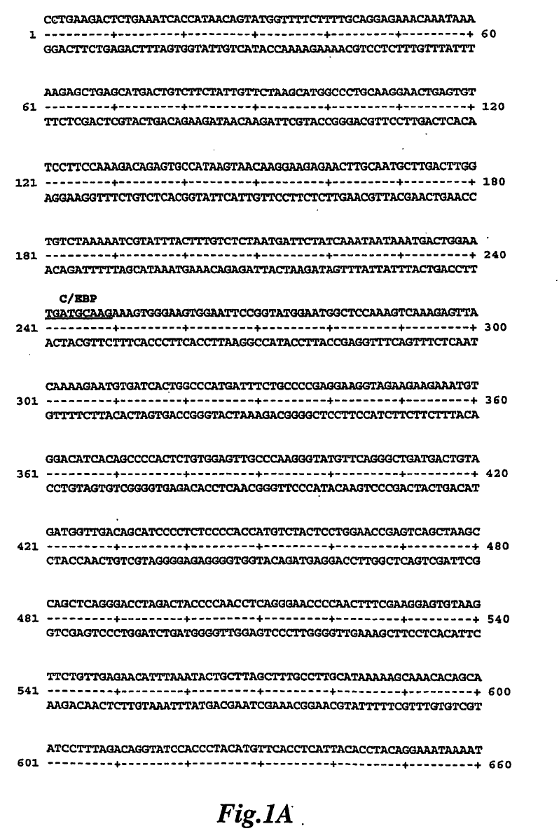

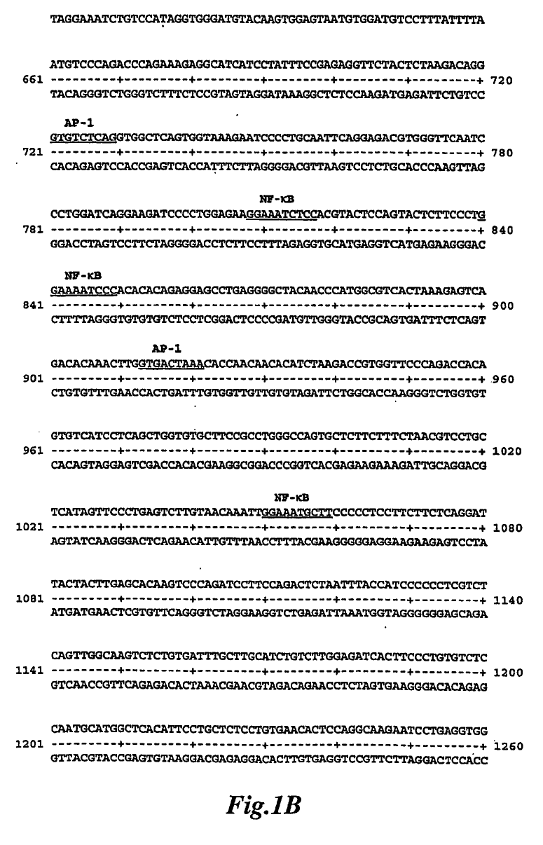

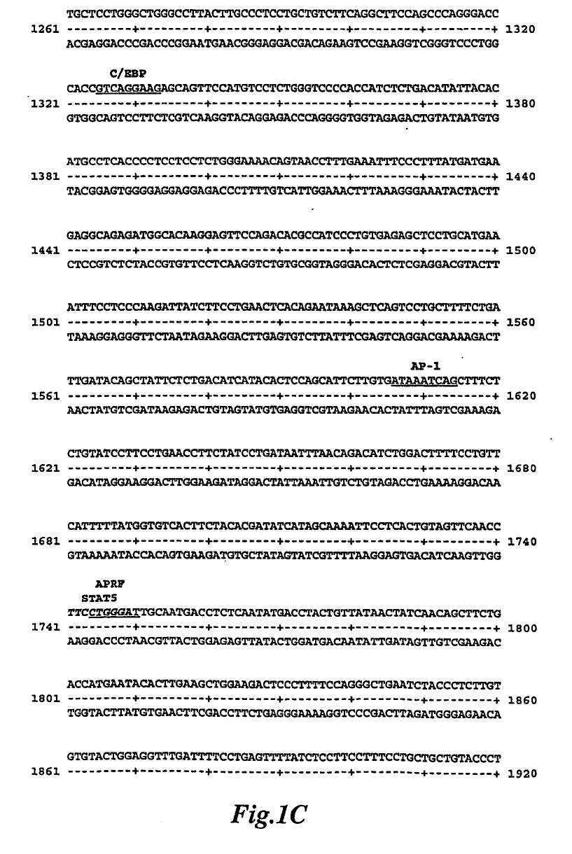

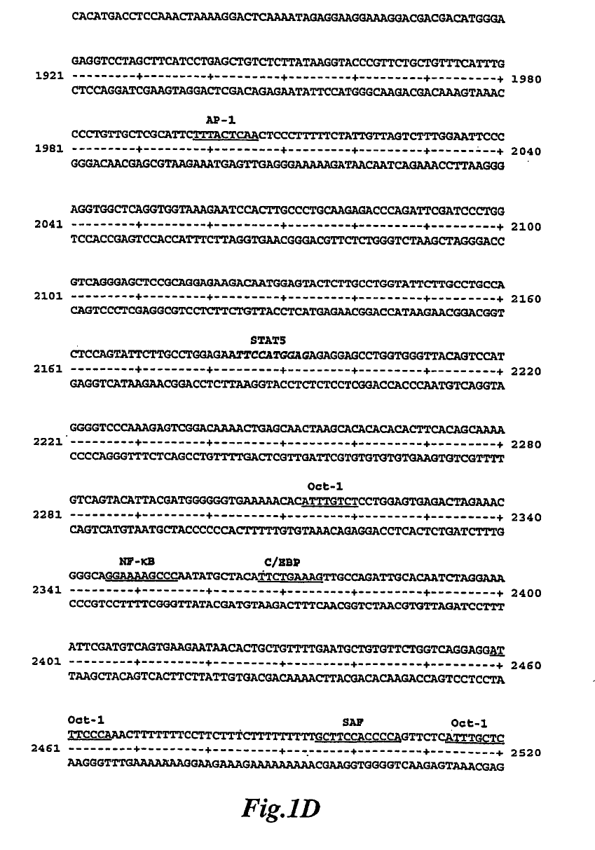

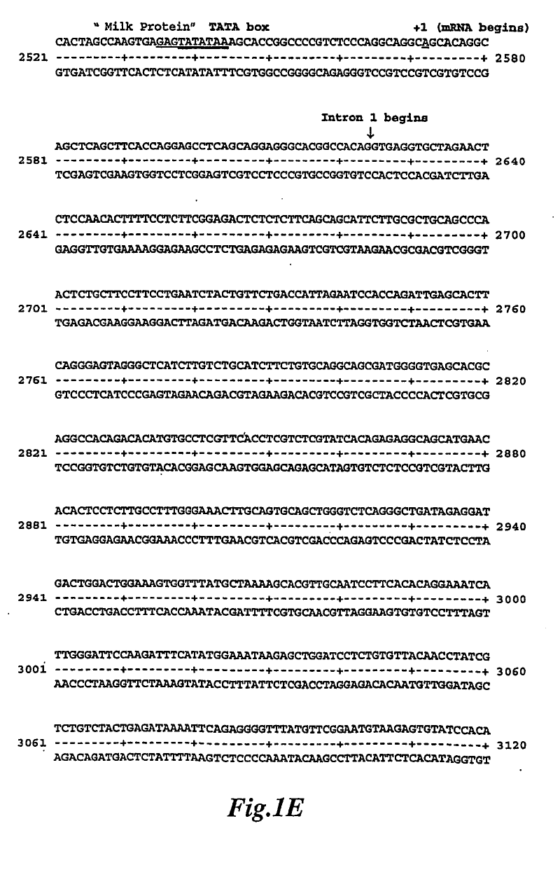

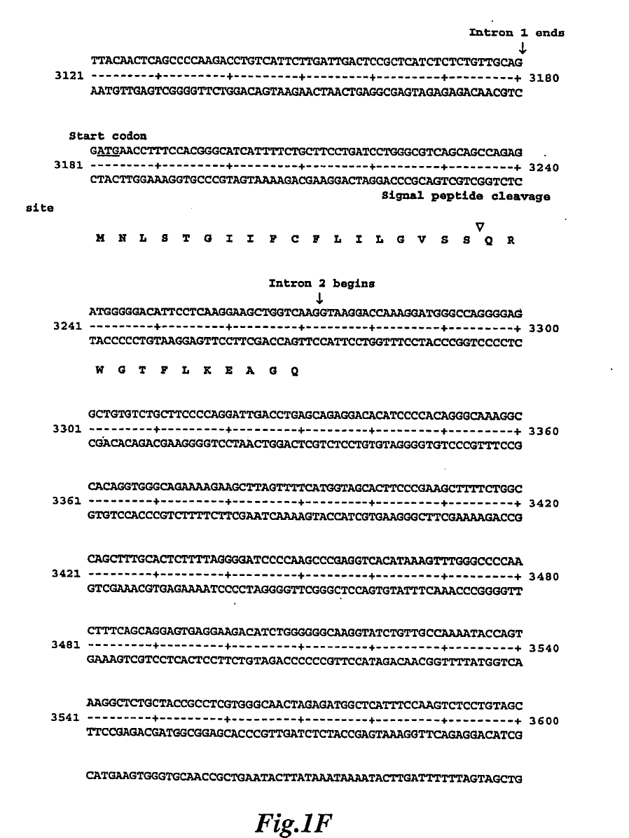

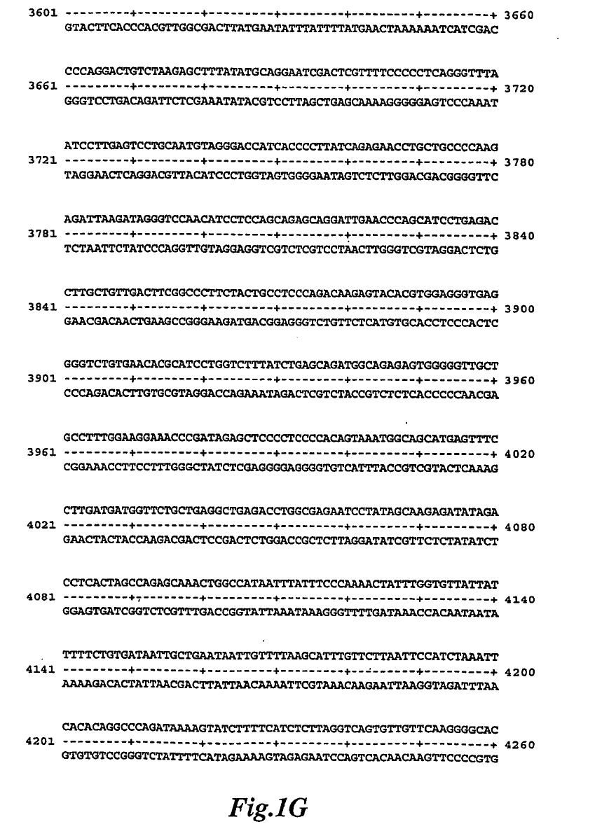

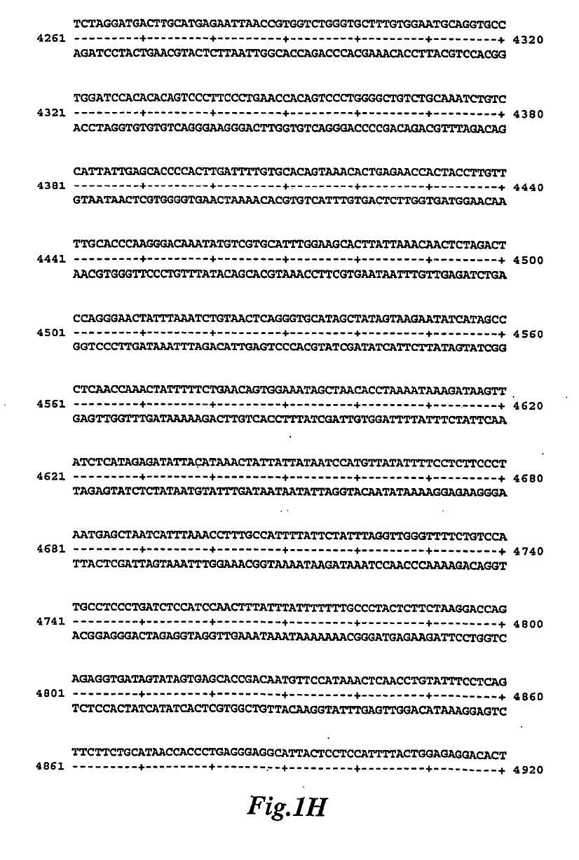

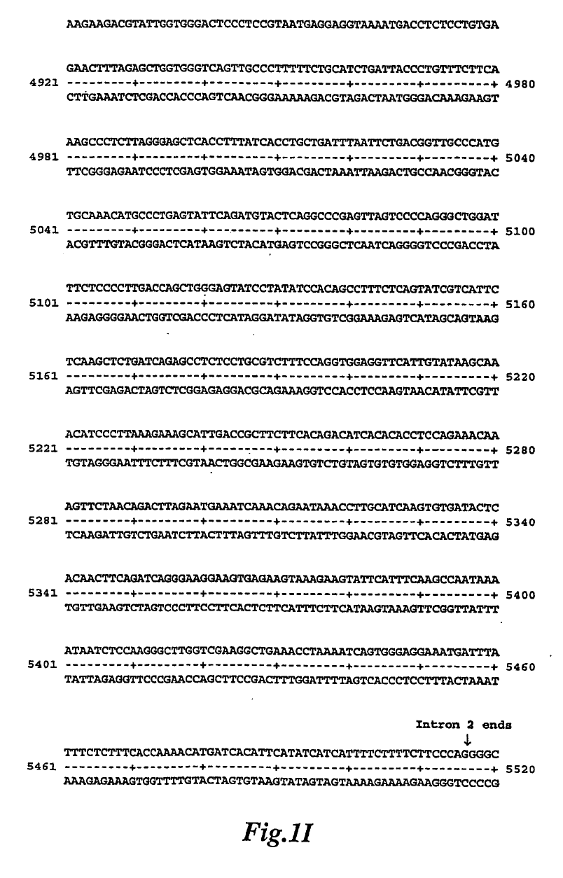

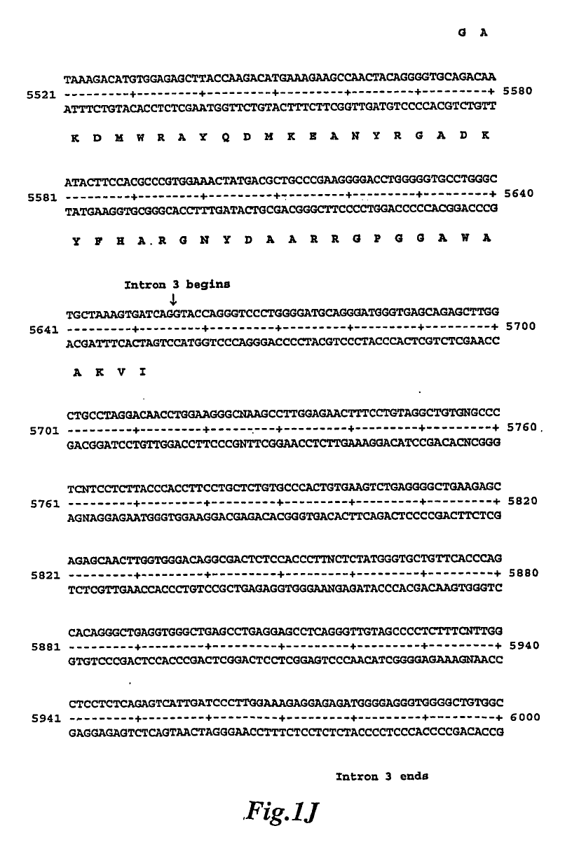

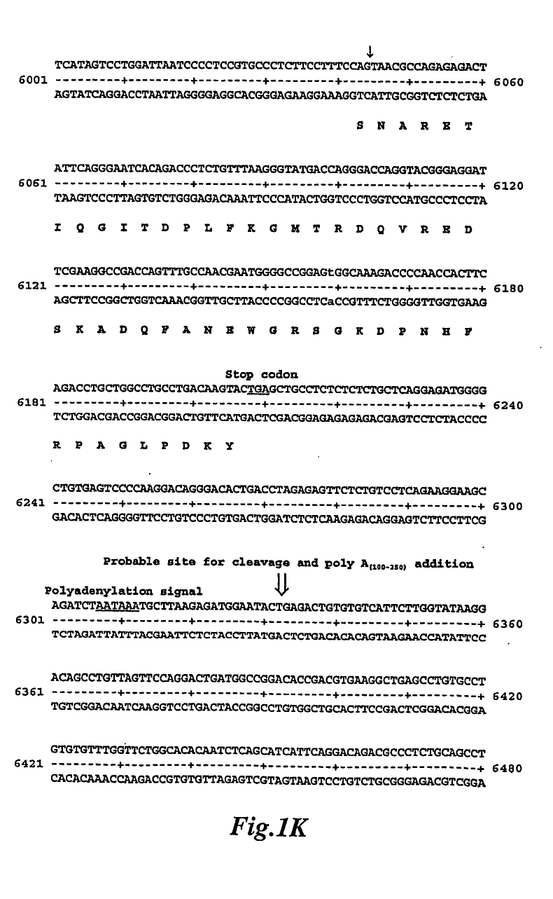

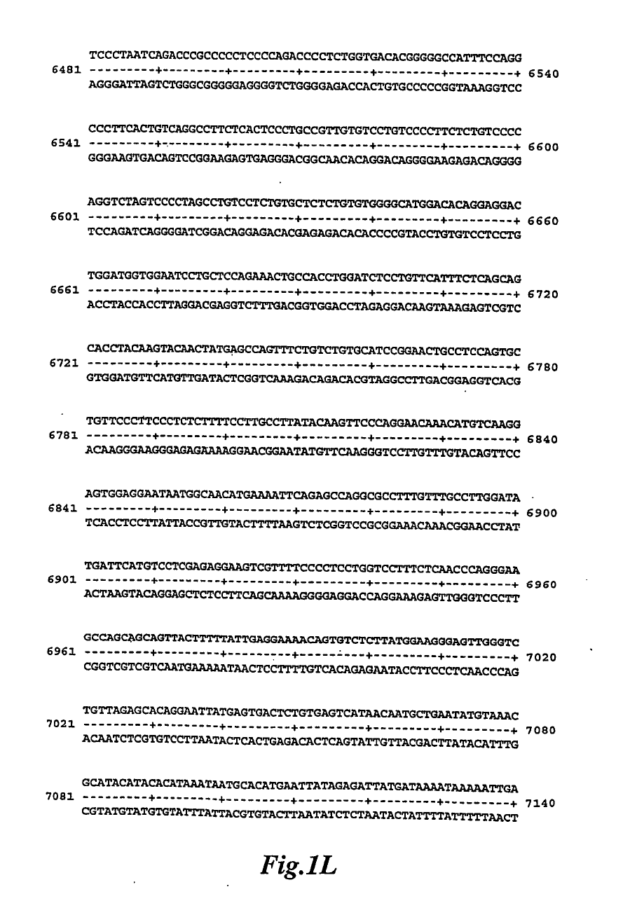

The resulting 2.4 kb StuI, 1.7 kb ScaI, 1.8 kb HincII, and 1.5 kb SspI secondary PCR products obtained from genomic walking, in addition to PCR products obtained using primer-walking methodology, were sequenced by the DNA Sequencing Facility at either Iowa State University or at the University of Nebraska Medical Center using the AP2 primer and/or M-SAA3 gene-specific primers. The DNA sequence from several independent high-fidelity PCR products was analyzed using the Wisconsin Genetics Computer Group (GCG) Package (Version 10.1, Madison, Wis.). Assembly of the overlapping amplicons provided the following nucleotide sequence for the promoter, introns, and 3′ flanking region of the bovine M-SAA3 gene (FIG. 1). The TATA box is double underlined in the promoter region for bovine mammary-associated serum amyloid A 3 (M-SAA3, MAA). The additional three upstream nucleotides (single underlined) from the TATA box are also conserved in most “milk protein” TATA boxes. Putative consensus STAT5 DNA-binding sites at −1741 to −1749 and at −2184 to −2193 are italicized in bold type and denoted above the sequence. Other possible transcription factor DNA-binding sites are underlined and the corresponding transcription factor for this consensus sequence is identified above the sequence (MatInspector V2.2/TRANSFAC 4.0, Quandt et al., 1995). The transcriptional start site is underlined and indicated above the nucleotide with +1. The beginning and ending of the three introns are denoted with an arrow above these regions. The start and stop codons are underlined and indicated above the nucleotides. The encoded amino acids are denoted under the double-stranded DNA sequence. The presumed signal peptide cleavage site to remove the leader sequence predicted by the SignalP program (Version 1. 1) (Nielsen et al, 1997) with 100% certainty is denoted by an inverted triangle. The polyadenylation signal (−6307 to −6312) is underlined and the probable site for cleavage and polyadenylation is indicated with a double arrow.

References useful in this example include the following:

Nielsen H., Engelbrecht J., Brunak S., von Heijne G. 1997. Identification of prokaryotic and eukaryotic signal peptides and prediction of their cleavage sites. Protein Engineering 10:1-6; Quandt K., Frech K., Karas H., Wingender E., Werner T. 1995. MatInd and MatInspector-New fast and versatile tools for detection of consensus matches in nucleotide sequence data. Nucleic Acids Research 23:4878-4884.

1. A purified and isolated mammary associated amyloid A promoter nucleotide sequence.

2. The promoter of claim 1 wherein said promoter sequence is isolated from and is natively associated with a bovine mammary associated amyloid A encoding gene sequence.

3. The promoter of claim 2 wherein said promoter nucleotide sequence is obtained from SEQ ID NO: 1.

4. The promoter of claim 3 wherein said promoter comprises bases 1-2571 of SEQ ID NO: 1 or its conservatively modified variants.

5. The promoter of claim 1 wherein said promoter nucleotide sequence is induced by prolactin.

6. The promoter of claim 1 wherein said promoter nucleotide sequence is induced by LPS.

7. The promoter of claim 1 wherein said promoter causes transcription and/or expression of operably linked nucleotide sequences in a bovine mammary epithelial cell.

8. A nucleotide construct comprising a nucleotide sequence the transcription of which is desired in a cell operably linked to a promoter region from bovine colostrum associated SAA.

9. The nucleotide construct of claim 8 wherein said nucleotide sequence is a protein encoding sequence.

10. The nucleotide construct of claim 9 wherein said sequence is an expression construct.

11. A vector comprising the nucleotide construct of claim 8.

12. A host cell transformed with the vector of claim 8.

13. A method of expressing a polypeptide in a cell comprising: providing to said cell a nucleic acid construct comprising a mammary amyloid A promoter operably linked to a nucleotide sequence encoding said peptide said promoter comprising:
(a) a promoter sequence obtained from SEQ ID NO: 1, or
(b) a sequence hybridizing under conditions of high stringency to the promoter in a above and; obtaining expression of said polypeptide in a cell.

14. The method of claim 13 wherein said nucleic acid construct is comprised within a vector.

15. The method of claim 14 wherein said vector is a viral vector.

16. A method of treating a disease associated with microbial infection in animals comprising: administering to said animal an effective amount of a mammary amyloid A promoter agonist.

17. The method of claim 16 wherein said agonist is prolactin.

18. The method of claim 16 wherein said agonist is LPS.

19. The method of claim 17 wherein said microbial infection is an enteric infection.

20. The method of claim 17 wherein site of microbial infection is mammary gland.

21. A method of treating a condition associated with enteric infection in animals comprising: administering to said animal an effective amount of a mammary amyloid A promoter agonist so that mucin production is stimulated.

22. The method of claim 1 wherein said enteric infection is selected from the group consisting of: Dysentery, prevention of infant diarrhea, traveler's diarrhea, necrotizing enterocolitis, and urinary tract infection.

23. A method of treating a disease associated with MAA underproduction in animals comprising: administering to said animal an effective amount of a mammary amyloid A promoter agonist.

24. A screening assay for identifying a composition which will increase or decrease MAA production and thereby treat diseases associated therewith comprising: providing said composition to a cell comprising the nucleotide construct of claim 8 and; assaying for MAA expression.

25. A method of treating diseases associated with mammary tissue microbial infection in mammals comprising: administering to said mammal an agent which stimulates production of colostrum associated SAA.

26. A method of treating diseases associated with mastitis in mammals comprising: administering to said mammal an agent which stimulates production of colostrum associated SAA.

27. The method of claim 25 wherein said agent is prolactin.

28. The method of claim 25 wherein said agent is LPS.
